(19) United States
(12) Patent Application Publication
(10) Pub. No.: US 2016/0257710 A1
(43) Pub. Date: Sep. 8, 2016

(54) SELECTIVE INHIBITORS OF PROTEINASE 3

(71) Applicants: INSERM (INSTITUT NATIONAL DE LA SANTÉ ET LA RECHERCHE MÉDICALE), Paris (FR); UNIVERSITE DE TOURS FRANCOIS RABELAIS, Tours (FR); UNIWERSYTET GDANSKI, Gdansk (PL)

(72) Inventors: Francis GAUTHIER, TOURS (FR); Brice KORKMAZ, TOURS (FR); Adam LESNER, Gdansk (PL)

(21) Appl. No.: 15/032,655

(22) PCT Filed: Nov. 4, 2014
(86) PCT No.: PCT/EP2014/073705

§ 371 Date: Apr. 28, 2016

(30) Foreign Application Priority Data
Nov. 4, 2013 (EP) 13306514.4
Apr. 11, 2014 (EP) 14305537.4

(51) Int. Cl. C07K 5/113; G01N 33/573; C07K 5/103; A61K 47/48; C07K 5/117

(52) U.S. Cl. CPC C07K 5/1021; A61K 47/48061; A61K 47/48215; C07K 5/1024; C07K 5/101; G01N 33/573; A61K 38/00

(57) ABSTRACT

The present invention relates to peptidyl phosphonate esters compounds and their use selective inhibitors of proteinase 3, in particular for treating or diagnosing inflammator autoimmune and cancer disorders. More specifically, the invention concerns nov peptidyl phosphonate esters compounds, including without limitation, compounds with Asp-Tyr-Asp-Ala or Pro-Tyr-Asp-Ala, Pro-Tyr-Asp-Avl, Val-Tyr-Asp-Avl peptide structure or their derivatives.

The present invention relates to peptidyl phosphonate esters compounds and their use as selective inhibitors of proteinase 3, in particular for treating or diagnosing inflammatory, autoimmune and cancer disorders. More specifically, the invention concerns novel peptidyl phosphonate esters compounds, including without limitation, compounds with Asp-Tyr-Asp-Ala, Pro-Tyr-Asp-Ala, Pro-Tyr-Asp-Avl or Val-Tyr-Asp-Avl peptide structure or their derivatives.

Though about 200 serine proteases have been identified in human, most of them remain poorly characterized with respect to their physiological substrates and function. They are recognized however as relevant molecular targets in a large number of diseases but a relatively low number of selective inhibitor that might modulate their proteolytic activity in vivo has been approved for clinical use (reviewed in (1)). Human proteinase 3 (PR3), a neutrophilic serine proteinase, shares many structural and functional characteristics with human neutrophil elastase (HNE). These proteinases are stored in huge amount in intracellular primary granules and contribute to the degradation of the extracellular matrix observed in infectious and inflammatory diseases especially in the lung. PR3 has also been identified as the principal antigen in autoimmune vasculitis (granulomatosis with polyangiitis (GPA), (formerly Wegener disease) (2-4) and most probably participate in the mechanism of apoptosis (5, 6). Unlike HNE, PR3 is also present in secretory vesicles in addition to primary granules, and is associated with the inner surface of the plasma membrane (7). Further, PR3 is the only neutrophilic serine protease to be constitutively expressed at the surface of circulating neutrophils (7). This genetically determined constitutive expression is probably related to its function of autoantigen in vasculitides (8). But all physiological inhibitors of PR3 reported so far, preferentially target HNE. This seriously complicates the investigation and understanding of its biological function (9).

Selective inhibitors of PR3 have been described. A substrate based approach has been used to convert SerpinB1 into a selective inhibitor of PR3 (20). Selective reversible azapeptide inhibitor of human neutrophil PR3derived from high affinity FRET substrate has also been described (21).

Peptidyl-diphenyl phosphonate inhibitors are transition state irreversible inhibitors that form a tetrahedral adduct with the seryl residue within the protease active site (10). They selectively inhibit serine proteases, are chemically stable in buffer and plasma in acidic and neutral conditions, and are effective at low concentrations (11). Further they can be used as activity-based probes to label serine proteases at the cell surface (12) or possibly inside the cell (13). A number of peptide diphenyl phosphonate inhibitors has been developed. These inhibitors very efficiently target HNE with kobs/I values in the 10³-10⁴ M⁻¹s⁻¹ range. But so far none are more potent toward PR3 than HNE (14).

Therefore, there is still a need to identify selective, irreversible and easy to handle PR3 inhibitors that could be used as activity-based probes able to detect specifically PR3 at the cell surface or inside cells. More specifically, there is a need to identify inhibitors capable of crossing the cell membrane and entering the cells. Such inhibitors may be valuable for use in diagnostic assays and for the development of anti-inflammatory drugs.

The inventors have now identified such selective inhibitors of proteinase 3.

### SUMMARY OF THE INVENTION

In a first aspect, the present invention provides a peptidyl phosphonate ester compound having the following structure of formula (I):

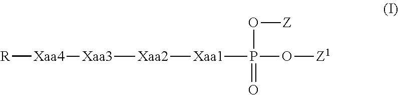

or a pharmaceutically acceptable salt thereof, wherein

Z and Z¹ are the same or different and are selected from the group consisting of C₁₋₆ perfluoralkyl, phenyl, phenyl substituted with J, phenyl disubstituted with J, phenyl trisubstituted with J, and, pentafluorophenyl,

J is selected from the group consisting of halogen, S, C₁₋₆ alkyl, C₁₋₆perfluoroalkyl, C₁₋₆ alkoxy, NO₂, CN, OH, CO₂H, amino, C₁₋₆alkylamino, C₂₋₁₂ dialkylamino, C₁₋₆ acyl, and C₁₋₆alkoxy-CO—, and C₁₋₆alkly-S—,

R is H, or is selected from the group consisting of protecting group, hydrophobic group and acetyl,

Xaa4 is an amino acid selected from the group consisting of proline, valine or any hydrophobic amino acid, or a negatively charged amino acid compound,

Xaa3 is any amino acid except alanine, for example selected from the group consisting of glycine, tyrosine, valine, leucine, isoleucine, proline, methionine, phenylalanine, tryptophane, serine, threonine, cysteine, asparagine, glutamine, aspartic acid, glutamic acid, lysine, arginine, histidine, phenylglycine, norleucine, norvaline, ornithine, citrulline,

Xaa2 is an amino acid selected from the group consisting of negatively charged aminoacid compound, tyrosine, phenylalanine and serine,

Xaa1 is alanine, 2-aminobutyric acid (Abu), valine or norvaline (Avl) or an equivalent non-polar and non-charged amino acid.

In specific embodiments, Z and Z¹ are selected from the group consisting of phenyl, phenyl substituted with J, and phenyl disubstituted with J. For example, Z and Z¹ may be 4-chlorophenyl.

In another specific embodiment, R is a non-polar aliphatic group or aryl group, and preferably R is either (i) a linear or branched alkyl chain, preferably (C5-C10) linear or branched alkyl chain, and their esters, ethers or polyethers or (ii) one or more hydrophobic amino acids selected from the group consisting of alanine, isoleucine, leucine, methionine, phenylalanine, proline, tryptophane and valine. In a specific embodiment, R is glycine.

In another specific embodiment, R is selected from the group consisting of biotin, polyethyleneglycol (PEG), and a fluorescent agent, optionally covalently coupled to the tetrapeptide Xaa4-Xaa3-Xaa2-Xaa1 indirectly via a spacer.

In preferred embodiments, Xaa4 is proline and Xaa2 is aspartic acid. In preferred embodiments, Xaa4 is proline, Xaa3 is tyrosine, Xaa2 is aspartic acid and Xaa1 is alanine. In other preferred embodiments, Xaa4 is proline, Xaa3 is tyrosine, Xaa2 is aspartic acid and Xaa1 is norvaline. In other preferred embodiments, Xaa4 is valine Xaa3 is tyrosine, Xaa2 is aspartic acid and Xaa1 is alanine. In other preferred embodiments, Xaa4 is valine, Xaa3 is tyrosine, Xaa2 is aspartic acid and Xaa1 is norvaline.

Examples of compounds according to the invention are:
i. Ac-Asp-Tyr-Asp-Ala^{P}(O—C₆H₄-4-Cl)₂
ii. Ac-Pro-Tyr-Asp-Ala^{P}(O—C₆H₄-4-Cl)₂;
iii. Biotin-Asp-Tyr-Asp-Ala^{P}(O—C₆H₄-4-Cl)₂;
iv. Biotin-Pro-Tyr-Asp-Ala^{P}(O—C₆H₄-4-Cl)₂;
v. Biotin-Pro-Tyr-Asp-Avl^{P}(O—C₆H₄-4-Cl)₂
vi. Biotin-Val-Tyr-Asp-Ala^{P}(O—C₆H₄-4-Cl)₂
vii. Biotin-Val-Tyr-Asp-Avl^{P}(O—C₆H₄-4-Cl)₂
viii. Biotin-(PEG)-Pro-Tyr-Asp-Ala^{P}(O—C₆H₄-4-Cl)₂;
ix. Ac-Pro-Tyr-Phe-Ala^{P}(O—C₆H₄-4-Cl)₂;
x. CH₃(CH₂)₄—Pro-Tyr-Asp-Ala^{P}(O—C₆H₄-4-Cl)₂;
xi. Ac-Ahx-Pro-Tyr-Asp-Ala^{P}(O—C₆H₄-4-Cl)₂;
xii. ⁺H₂N-Pro-Tyr-Asp-Ala^{P}(O—C₆H₄-4-Cl)₂;
xiii. Ac-Trp-Pro-Tyr-Asp-Ala^{P}(O—C₆H₄-4-Cl)₂; and,
xiv. Ac-Ile-Pro-Tyr-Asp-Ala^{P}(O—C₆H₄-4-Cl)₂,
   or their pharmaceutically acceptable salts.

Said compounds can be used as selective inhibitors of PR3, in particular as a drug. The compounds are useful for example in treating disorders affected by abnormal or pathological activity of PR3. Such disorders include, without limitation, infectious, autoimmune and inflammatory disorders, characterized by neutrophilic inflammation, such as cystic fibrosis, chronic obstructive pulmonary disorder (COPD), acute lung injury, granulomatosis with polyangiitis, rheumatoid arthritis, and all forms of cancer that associate a neutrophil recruitment.

The compounds according to the invention may also be useful in diagnostics, as a marker for PR3.

The invention further relates to a pharmaceutical composition, comprising a compound according to the invention as defined above, and one or more pharmaceutically acceptable vehicle or excipient.

It is a further object of the invention to provide in vitro methods for detecting or quantifying PR3 in a biological sample, comprising the steps of
a. providing a biological sample;
b. contacting said biological sample with a compound of the invention under conditions for specific binding of said compound with PR3; and,
c. detecting specific binding of said compound with PR3;
   wherein said specific binding enables determining the presence or amount of PR3 in said biological sample.

Preferably in such diagnostic methods, said biological sample is from a human suffering from disorders selected from the group consisting of infection, autoimmune and inflammatory disorders, characterized by neutrophilic inflammation, such as cystic fibrosis, COPD, acute lung injury, granulomatosis with polyangiitis, rheumatoid arthritis, and cancers associated with neutrophil recruitment.

### DETAILED DESCRIPTION

### Definitions

As used herein, the term “amino acid” refers to natural or unnatural amino acids in their D and L stereoisomers for chiral amino acids. It is understood to refer to both amino acids and the corresponding amino acid residues, such as are present, for example, in peptidyl structure. Natural and unnatural amino acids are well known in the art. Common natural amino acids include, without limitation, alanine (Ala), arginine (Arg), asparagine (Asn), aspartic acid (Asp), cysteine (Cys), glutamine (Gin), glutamic acid (Glu), glycine (Gly), histidine (His), isoleucine (Ile), leucine (Leu), Lysine (Lys), methionine (Met), phenylalanine (Phe), proline (Pro), serine (Ser), threonine (Thr), tryptophan (Trp), tyrosine (Tyr), and valine (Val). Uncommon and unnatural amino acids include, without limitation, allyl glycine (AllyIGly), norleucine, norvaline (Avl), biphenylalanine (Bip), citrulline (Cit), 4-guanidinophenylalanine (Phe(Gu)), homoarginine (hArg), homolysine (hLys), 2-naphtylalanine (2-Nal), ornithine (Orn) and pentafluorophenylalanine.

Amino acids are typically classified in one or more categories, including polar, hydrophobic, acidic, basic and aromatic, according to their side chains. Examples of polar amino acids include those having side chain functional groups such as hydroxyl, sulfhydryl, and amide, as well as the acidic and basic amino acids. Polar amino acids include, without limitation, asparagine, cysteine, glutamine, histidine, selenocysteine, serine, threonine, tryptophan and tyrosine. Examples of hydrophobic or non-polar amino acids include those residues having nonpolar aliphatic side chains, such as, without limitation, leucine, isoleucine, valine, glycine, alanine, proline, methionine and phenylalanine. Examples of basic amino acid residues include those having a basic side chain, such as an amino or guanidino group. Basic amino acid residues include, without limitation, arginine, homolysine and lysine. Examples of acidic amino acid residues include those having an acidic side chain functional group, such as a carboxy group. Acidic amino acid residues include, without limitation aspartic acid and glutamic acid. Aromatic amino acids include those having an aromatic side chain group. Examples of aromatic amino acids include, without limitation, biphenylalanine, histidine, 2-napthylalananine, pentafluorophenylalanine, phenylalanine, tryptophan and tyrosine. It is noted that some amino acids are classified in more than one group, for example, histidine, tryptophan and tyrosine are classified as both polar and aromatic amino acids.

Amino acids may further be classified as non-charged, or charged (positively or negatively) amino acids. Examples of positively charged amino acids include without limitation lysine, arginine and histidine. Examples of negatively charged amino acids include without limitation glutamic acid and aspartic acid. Additional amino acids that are classified in each of the above groups are known to those of ordinary skill in the art.

“Equivalent amino acid” means an amino acid which may be substituted for another amino acid in the peptide compounds according to the invention without any appreciable loss of function. Equivalent amino acids will be recognized by those of ordinary skill in the art. Substitution of like amino acids is made on the basis of relative similarity of side chain substituents, for example regarding size, charge, hydrophilicity and hydrophobicity as described herein. The phrase “or an equivalent amino acid thereof” when used following a list of individual amino acids means an equivalent of one or more of the individual amino acids included in the list.

As used herein, the term “Ahx” stand for the derivative of lysine amino acid, 6-(aminohexanoic) acid.

The term “ester” is represented by the formula —OC(O)R, where R can be an alkyl, alkenyl, alkynyl, aryl, aralkyl, cycloalkyl, halogenated alkyl, or heterocycloalkyl group described above.

The term “aliphatic group” is defined as including branched or unbranched alkyl, alkenyl, alkynyl, halogenated alkyl and cycloalkyl groups.

The term “alkyl group” refers to a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl, and the like. A “lower alkyl group” has from 1 to 10 carbon atoms.

The term “alkenyl group” refers to hydrocarbon group of 2 to 24 carbon atoms and structural formula containing at least one carbon-carbon double bond.

The term “alkynyl group” refers to hydrocarbon group of 2 to 24 carbon atoms and structural formula containing at least one carbon-carbon triple bond.

The term “halogen” refers to F, Cl, Br or I. A “halogenated alkyl group” or “haloalkyl” refer to an alkyl group as defined above with one or more hydrogen atoms present on these groups substituted with a halogen.

The term “aryl group” refers to any carbon-based aromatic group including, but not limited to, benzene, naphtalene. The term “aryl” further includes heteroaryl group which is defined as an aromatic group that has at least one heteroatom incorporated within the ring of the aromatic group, such heteroatoms, including, without limitation, nitrogen, oxygen, sulfur, and phosphorous. The aryl groups can also be substituted with one or more groups including, but not limited to, alkyl, alkynyl, alkenyl, aryl, halide, nitro, amino, ester, ketone, aldehyde, hydroxy, carboxylic acid, or alkoxy.

As used herein, polyethyleneglycol (PEG) refers to any polymers of ethyleneglycol of the following formula:

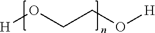

or its derivatives,
wherein n is at least superior to 4 and its molecular weight is preferably comprised between 400 and 5000 Da.

The term “Proteinase 3” refers to the serine protease enzyme, also referred as PR3 or PRTN3, and expressed mainly in neutrophil granulocytes. In human neutrophils, PR3 contributes to the proteolytic generation of antimicrobial peptides. It is also the epitope of anti-neutrophil cytoplasmic antibodies (ANCAs) of the c-ANCA (cytoplasmic subtype) class, a type of antibody frequently found in the disease granulomatosis with polyangiitis (GPA) (formerly Wegener's granulomatosis). In a specific embodiment, proteinase 3 refers to human proteinase 3 of UNIPROT accession number P24158.

The term “human neutrophil elastase” refers to serine protease enzyme expressed in neutrophil, also referred as HNE. In a specific embodiment, human neutrophil elastase refers to the protein of UNIPROT accession number P08246.

### Proteinase 3 Inhibitors of the Invention

The invention relates to novel derivatives of peptidyl phosphonate esters compounds, which are selective inhibitors of PR3.

Peptidyl phosphonate esters are analogues of natural α-amino acids and are designated by the generally accepted three letter abbreviations for the amino acid followed by the superscript P. They inhibit serine protease by reaction with active site serine to form “phosphonylated” enzymes, which due to the similarity of phosphorus atom to the tetrahedral intermediate formed during peptide hydrolysis show remarkable stability.

Examples of peptidyl phosphonate esters compounds and their synthesis have already been described in U.S. Pat. No. 5,543,396 and Boduszek et al, 1994 (11).

The PR3 inhibitors of the invention include a peptidyl phosphonate ester compound having the following structure of formula (I):

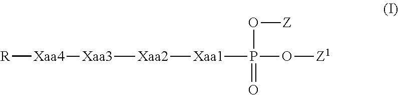

or a pharmaceutically acceptable salt thereof.

In formula (I), Z and Z¹ are the same or different and are selected from the group consisting of C₁₋₆ perfluoralkyl, phenyl, phenyl substituted with J, phenyl disubstituted with J, phenyl trisubstituted with J, and, pentafluorophenyl. J is selected from the group consisting of halogen, S, C₁₋₆ alkyl, C₁₋₆perfluoroalkyl, C₁₋₆ alkoxy, NO₂, CN, OH, CO₂H, amino, C₁₋₆alkylamino, C₂₋₁₂ dialkylamino, C₁₋₆ acyl, and C₁₋₆alkoxy-CO—, and C₁₋₆alkly-S—.

In certain specific embodiments, J is chlorure or fluorure. In related specific embodiments, Z and Z¹ are 4-chlorophenyl or 4-fluorophenyl.

In specific embodiments, R may be absent or is an acetyl group. In other embodiments, R is a protecting group. Protecting groups which may be used are those well known in the Art of peptide synthesis. Suitable protecting groups may be selected from the group consisting of aminobenzyl (Abz), 9-fluorenylometoxy (Fmoc), carbobenzyloxy (Cbz), benzoyl, t-butyloxycarbonyl (t-Boc), glutaryl, p-tolylsulfonyl (Tos), methoxysuccinyl (MeO-Suc), and succinyl. Alternative protecting groups include polyethylene glycol derivatives.

In other specific embodiments, R is a hydrophobic group, for example selected from non-polar aliphatic groups or aryl groups, preferably with at least 5 carbon atoms. In specific embodiment, R is selected from linear non-polar aliphatic chains, branched or unbranched, saturated or not saturated, having between 5 and 24 carbon atoms. For example, R is biotin, biotin-(PEG), or a non-polar linear or branched C5-C10 alkyl chain, for example CH₃—(CH₂)₄—, acetyl hexanoic acid, and linear polyethers.

In other specific embodiments, R is a hydrophobic amino acid, for example selected from the group consisting of leucine, isoleucine, valine, proline, methionine and phenylalanine. In another specific embodiment, R is glycine.

In other specific embodiments, R is an α-amino acid equivalent to the P₅ residue of PR3 specific substrates, for example selected from the group consisting of isoleucine.

R may also be any covalently bound molecule, optionally via a spacer, for functionalization or vectorization of the compounds according to the invention. For example, for use in diagnostic methods, R may be a labeling agent, including fluorescent agents or biotin. In specific embodiments, said labeling agent or biotin is covalently bound to the compounds via a spacer selected from the group consisting of PEG (e.g. PEG3000) or any linear aliphatic chains.

In formula (I), Xaa1, Xaa2, Xaa3 and Xaa4 may represent amino acid residues of the peptide structures of the inhibitors. The tetrapeptide sequence Xaa4-Xaa3-Xaa2-Xaa1 may reflect for example, the P₄-P₃-P₂-P₁ residues of PR3 specific substrates.

In certain specific embodiments, one or more of the amide linkages of the tetrapeptides may be substituted by an ester linkage to produce pseudopeptide phosphonate inhibitors. Methods for producing pseudopeptide phosphonate compounds are described for example in Bioorganic & medicinal chemistry 2011, 19, 1277-1284. In a preferred embodiment, a pseudopeptide is made by substituting the amide linkage between Xaa2 and Xaa3 by an ester linkage.

In specific embodiments, Xaa4 is an amino acid selected from the group consisting of proline, valine or any hydrophobic amino acid, or negatively charged aminoacid compound, for example glutamic acid or aspartic acid or an equivalent amino acid residue. Xaa3 is any amino acid except alanine. For example, Xaa3 is selected from the group consisting of: glycine, tyrosine, valine, leucine, isoleucine, proline, methionine, phenylalanine, tryptophane, serine, threonine, cysteine, asparagine, glutamine, aspartic acid, glutamic acid, lysine, arginine, histidine, phenylglycine, norleucine, norvaline, ornithine, citrulline. Preferably, Xaa3 is tyrosyine (Tyr) or an equivalent amino acid residue. Xaa2 is a negatively charged aminoacid compound, for example selected from the group consisting of aspartic acid or glutamic acid or an equivalent amino acid residue, preferably aspartic acid. Xaa1 is a non-polar or non-charged amino acid, preferably alanine or valine or, more preferably norvaline, or an equivalent amino acid residue. In preferred embodiments, Xaa4 is proline and Xaa2 is Asp. In another specific embodiment, Xaa4 is proline, Xaa3 is Tyr, Xaa2 is Asp and Xaa1 is Ala. In another specific embodiment, Xaa4 is proline, Xaa3 is Tyr, Xaa2 is Asp and Xaa1 is Ala and R is a non-polar aliphatic group, preferably (C5-C10) non-polar aliphatic group. In other preferred embodiments, Xaa4 is valine and Xaa2 is Asp. In another specific embodiment, Xaa4 is valine, Xaa3 is Tyr, Xaa2 is Asp and Xaa1 is Ala or Avl. In another specific embodiment, Xaa4 is valine, Xaa3 is Tyr, Xaa2 is Asp and Xaa1 is Ala or Avl and R is a non-polar aliphatic group, preferably (C5-C10) non-polar aliphatic group.

In certain embodiments, the tetrapeptide structure of the compound is selected from the group consisting of: Asp-Tyr-Asp-Ala, Pro-Xaa3-Tyr-Xaa1, Pro-Tyr-Asp-Ala, Val-Tyr-Asp-Ala, Pro-Tyr-Asp-Avl, Val-Tyr-Asp-Avl and related peptides or derivatives with equivalent amino acid residues.

Working examples of the compounds of the invention include the following compounds:
Compound 1: Ac-Asp-Tyr-Asp-Ala^{P}(O—C₆H₄-4-Cl)₂;
Compound 2: Ac-Pro-Tyr-Asp-Ala^{P}(O—C₆H₄-4-Cl)₂;
Compound 3: Biotin-Asp-Tyr-Asp-Ala^{P}(O—C₆H₄-4-Cl)₂;
Compound 4: Biotin-Pro-Tyr-Asp-Ala^{P}(O—C₆H₄-4-Cl)₂;
Compound 5: Biotin-Pro-Tyr-Asp-Avl^{P}(O—C₆H₄-4-Cl)₂;
Compound 6: Biotin-Val-Tyr-Asp-Ala^{P}(O—C₆H₄-4-Cl)₂;
Compound 7: Ac-Pro-Tyr-Phe-Ala^{P}(O—C₆H₄-4-Cl)₂;
Compound 8: CH₃(CH₂)₄-Pro-Tyr-Asp-Ala^{P}(O—C₆H₄-4-Cl)₂;
Compound 9: Ac-Ahx-Pro-Tyr-Asp-Ala^{P}(O—C₆H₄-4-Cl)₂;
Compound 10: ⁺H₂N-Pro-Tyr-Asp-Ala^{P}(O—C₆H₄-4-Cl)₂;
Compound 11: Ac-Trp-Pro-Tyr-Asp-Ala^{P}(O—C₆H₄-4-Cl)₂;
Compound 12: Ac-Ile-Pro-Tyr-Asp-Ala^{P}(O—C₆H₄-4-Cl)₂;
Compound 13: Biotin-PEG₃₀₀₀-Pro-Tyr-Asp-Ala^{P}(O—C₆H₄-4-Cl)₂.

The compounds of the invention also encompass salts, including, if several salt-forming groups are present, mixed salts and/or internal salts. The salts are generally pharmaceutically acceptable salts that are non-toxic. These salts can include, for example, nontoxic metal cations which are derived from metals of groups IA, IB, IIA and IIB of the periodic table of the elements. In one embodiment, alkali metal cations such as lithium, sodium, or potassium ions, or alkaline earth metal cations such as magnesium or calcium can be used. The salt can also be a zinc or ammonium cation. The salt can also be formed with suitable organic amines, such as unsubstituted or hydroxyl-substituted mono-, di- or or tri-alkylamines, or with quaternary ammonium compounds.

### Selection of Preferred Compounds as Selective Inhibitors of Proteinase 3

The compounds of the invention are advantageously selective inhibitors of PR3. In particular, they can selectively inhibit PR3 while not inhibiting related human neutrophil elastase. As described under the Examples below (see PR3 inhibition assay), and as is generally known, efficacy of particular inhibitors against a target protease, for example, recombinant human PR3, is determined by combining the target with a substrate (for example Suc-AanV-SBzI), both in the absence of the inhibitor and in the present of different concentrations of such inhibitor to be tested. Typically, the efficacy of irreversible inhibitors are measured in terms of the second order rate constant of inhibition value (11) or kobs/l. Preferred inhibitors of the invention have typically a kobs/I of more than 10 M⁻¹s⁻¹, preferably more than 100 M⁻¹s⁻¹, and more preferably of more than 103 M⁻¹s⁻¹.

Preferably, the selective inhibitors according to the invention do not inhibit elastase enzymatic activity, for example as described in the protease inhibition assay under the Examples. In specific embodiments, a compound that does not inhibit elastase is a compound which exhibits a [l] value of more than 1 mM, and preferably, where no significant inhibition is detected.

By way of Example, compounds 1-13 were evaluated for PR3 and elastase inhibition and their [l] and kobs/[l] values are shown in Table 1:

TABLE 1

| | | [I] | Proteinase 3 | Elastase |
|---|---|---|---|---|
| | PEPTIDYL PHOSPHONATE ESTERS | μM | k_{obs}/[I] (M⁻¹s⁻¹) | |
| Compound 1 | Ac-Asp-Tyr-Asp-AlaP(O—C₆H₄-4-Cl)₂ | 200 | 1.9 ± 0.3 | n.s |
| Compound 2 | Ac-Pro-Tyr-Asp-AlaP(O—C₆H₄-4-Cl)₂ | 2 | 154 ± 3 | n.s |
| Compound 3 | Biotin-Asp-Tyr-Asp-AlaP(O—C₆H₄-4-Cl)₂ | 25 | 18 ± 2.8 | n.s |
| Compound 4 | Biotin-Pro-Tyr-Asp-AlaP(O—C₆H₄-4-Cl)₂ | 0.06 | 4168 ± 553 | n.s |
| Compound 5 | Biotin-Pro-Tyr-Asp-AvlP(O—C₆H₄-4-Cl)₂ | 0.008 | 33360 ± 4150 | n.s |
| Compound 6 | Biotin-Val-Tyr-Asp-AlaP(O—C₆H₄-4-Cl)₂ | 0.01 | 17396 ± 835 | n.s |
| Compound 7 | Ac-Pro-Tyr-Phe-AlaP(O—C₆H₄-4-Cl)₂ | 100 | 2.1 ± 0.2 | n.s |
| Compound 8 | CH3—(CH2)₄-Pro-Tyr-Asp-AlaP(O—C₆H₄-4-Cl)₂ | 0.05 | 4418 ± 329 | n.s |
| Compound 9 | Ac-Ahx-Pro-Tyr-Asp-AlaP(O—C₆H₄-4-Cl)₂ | 0.07 | 2159 ± 235 | n.s |
| Compound10 | ⁺H₂N-Pro-Tyr-Asp-AlaP(O—C₆H₄-4-Cl)₂ | 50 | 4.7 ± 0.5 | n.s |
| Compound 11 | Ac-Trp-Pro-Tyr-Asp-AlaP(O—C₆H₄-4-Cl)₂ | 15 | 15 ± 0.1 | n.s |
| Compound 12 | Ac-Ile-Pro-Tyr-Asp-AlaP(O—C₆H₄-4-Cl)₂ | 5 | 46 ± 0.1 | 15.1 ± 1.6 |
| Compound 13 | Biotin-PEG3000-Pro-Tyr-Asp-AlaP(O—C₆H₄-4-Cl)₂ | 0.2 | 1163 ± 0.1 | 46.5 |

### Pharmaceutical Compositions

Another aspect of the present invention includes pharmaceutical compositions prepared for administration to a subject and which include a therapeutically effective amount of one or more of the compounds of the invention, as described above. The therapeutically effective amount of a compound will depend on the route of administration, the type of mammal that is the subject and the physical characteristics of the subject being treated. Specific factors that can be taken into account include disease severity and stage, weight, diet and concurrent medications. The relationship of these factors to determining a therapeutically effective amount of the disclosed compounds is understood by those of ordinary skill in the art.

Any of the compounds described herein can be combined with a pharmaceutically acceptable vehicle or excipients to form a pharmaceutical composition. Pharmaceutical vehicle or excipients are known to those skilled in the art. These most typically would be standard vehicles or excipients for administration of compositions to humans, including solutions such as sterile water, saline, and buffered solutions at physiological pH. The compositions could also be administered intramuscularly, subcutaneously, or in an aerosol form. Other compounds will be administered according to standard procedures used by those skilled in the art. Pharmaceutical excipients can include thickeners, diluents, buffers, preservatives, surface active agents and the like in addition to the molecule of choice. Descriptions of some of these pharmaceutically acceptable excipients or vehicles may be found in The Handbook of Pharmaceutical Excipients, published by the American Pharmaceutical Association and the Pharmaceutical Society of Great Britain. Remington: the Science and Practice of Pharmacy 20th edition (2000), describes compositions and formulations suitable for pharmaceutical delivery of the compounds of the invention, in the form of aqueous solutions, lyophilized or other dried formulations. Pharmaceutical compositions can also include one or more additional active ingredients such as antimicrobial agents, anti-inflammatory agents, anti-cancer or anti-tumoral agents, anesthetics, and the like.

In general, the nature of the vehicle will depend on the particular mode of administration being employed. For instance, parenteral formulations usually contain injectable fluids that include pharmaceutically and physiologically acceptable fluids such as water, physiological saline, balanced salt solutions, aqueous dextrose, glycerol or the like as a vehicle.

In solid oral preparations, for example, powders, granules, capsules, caplets, gelcaps, pills and tablets (each including immediate release, timed release and sustained release formulations), suitable vehicles and excipients include but are not limited to diluents, granulating agents, lubricants, binders, glidants, disintegrating agents and the like. Because of their ease of administration, tablets and capsules represent the most advantageous oral dosage unit form, in which solid pharmaceutical excipients are obviously employed. If desired, tablets may be sugar coated, gelatin coated, film coated or enteric coated by standard techniques.

Preferably these compositions are in unit dosage forms, such as tablets, pills, capsules, powders, granules, lozenges, sterile parenteral solutions or suspensions, metered aerosol or liquid sprays, drops, ampoule, autoinjector devices, or suppositories for administration by oral, intranasal, sublingual, intraocular, transdermal, parenteral, rectal, vaginal or insufflation means.

In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

In a specific embodiment, the compositions are formulated for their administration into the airways, e.g. by inhalation. The pharmaceutical composition of the invention may thus be formulated as solution appropriate for inhalation.

### Therapeutic Methods for Using the Proteinase 3 Inhibitors of the Invention

The disclosed compounds can be used to selectively inhibit PR3, in vitro or in vivo and thus can be used in the prevention or treatment of conditions characterized by abnormal or pathological PR3 activity.

For example, the compounds are useful for prevention or treatment of infectious disorders, autoimmune and inflammatory disorders, characterized by neutrophilic inflammation. Autoimmune and inflammatory disorders characterized by neutrophilic inflammation include without limitation as cystic fibrosis, chronic obstructive pulmonary disorder (COPD), acute lung injury, granulomatosis with polyangiitis (GPA) and rheumatoid arthritis. Other disorders include, without limitation, pulmonary infectious diseases, pulmonary inflammatory conditions, asthma, pulmonary emphysema, bronchitis, psoriasis, allergic rhinitis, viral rhinitis, glomerulonephritis, postoperative adhesion formation, and reperfusion injury.

The compounds are also useful for prevention and treatment of cancer associated with neutrophil recruitment. Typical examples of cancers that may be treated according to the compounds of the invention include bladder and lung cancer.

Therefore, in one specific embodiment, the invention relates to a method for treating inflammatory and other disorders characterized by abnormal or pathological PR3 activity in a subject in need thereof, comprising administering to the subject a therapeutically effective amount of any of the compounds or compositions described above. Also included is the use of a compound of formula (I) for the preparation of a medicament for treating inflammatory and other disorders characterized by abnormal or pathological PR3 activity in a subject in need thereof. The term “treatment” as used herein refers to methods of improving, halting, retarding, preventing or palliating said disorders or at least one symptom of said disorders in the subject in need thereof.

The subject typically will be a mammal, and more specifically a human. The compounds of the invention may be administered by any pharmaceutically acceptable means, by either systemic or local administration. Modes of administration include oral, dermal, such as using a transdermal patch, inhalation, infusion, intranasal, rectal, vaginal, topical parenteral, including intraperitoneal, intravenous, intramuscular and subcutaneous injection. Pharmaceutically acceptable formulations and compositions are described above.

In one specific embodiment, the compounds are administered by inhalation. Any of the various means known in the art for administering therapeutically active agents by inhalation (pulmonary delivery) can be used in the methods of the present invention.

Such delivery methods are well-known in the art. Commercially available aerosolizers for liquid formulations, including jet nebulizers and ultrasonic nebulizers may be used. For delivery in liquid form, liquid formulation can be directly aerosolized and lyophilized powder can be aerosolized after reconstitution. For delivery in dry powder form, the formulation may be prepared as a lyophilized and milled powder. In addition, formulations may be delivered using a fluorocarbon formulation or other propellant and a metered dose dispenser. In specific embodiments, for example, nebulizers, which convert liquids into aerosols of a size that can be inhaled into the lower respiratory tract, are used, either in conjunction with a mask or a mouthpiece. In other embodiments, metered dose inhalers may be used. In yet other embodiments, dry powder delivery devices are also known and can be used.

The therapeutically effective amount of the compound or compounds administered can vary depending upon the desired effects and the factors noted above. In particular examples dosages are administered that achieve target tissue concentrations that have been found to be effective in vitro. Typically, dosages will be between about 0.01 mg/kg and 100 mg/kg of the subject's body weight, and more typically between about 0.05 mg/kg and 10 mg/kg, such as from about 0.2 to about 5 mg/kg of the subject's body weight.

The compounds of the invention may be used alone, in combination with other serine protease inhibitors, and more specifically HNE, cathepsin G (CatG) and/or chymase inhibitors, for example including SerpinB1 or α1-PI (9). The compounds of the invention may also be used in combination with other known anti-inflammatory agents or anti-cancer agents.

### Diagnostic or Screening Methods for Using the Proteinase 3 Inhibitors of the Invention

The compounds of the invention are also useful, for example, in in vitro assays, such as assays directed to identifying new inhibitors of PR3. The compounds can also be used to monitor or detect PR3 activity in biological samples, such as in a clinical situation from biopsy.

Typically, the compounds may be used in an in vitro method for detecting or quantifying PR3 in a biological sample, comprising the steps of
a. providing a biological sample;
b. contacting said biological sample with a compound of the invention, under conditions for specific binding of said compound with PR3; and,
c. detecting specific binding of said compound with PR3;
wherein said specific binding enables determining the presence or amount of PR3 in said biological sample.

The compounds of the invention may be labelled using conventional labelling agent or indirect markers which are then labelled by generic labelling agent for allowing detection of specific binding and/or quantifying PR3 or PR3 activity in the biological sample. A person skilled in the art will understand that a number of methods can be used to detect and/or quantify specific labelled compounds of the invention, including immunoassays such as Western Blots, ELISA, and immunoprecipitation followed by SDS-PAGE, as well as immunocytochemistry or immunohistochemistry, fluorescent or other light-emission detectors or analytical techniques.

Kits may be prepared for carrying out one of the above mentioned diagnostic methods. Thus, the invention further relates to a kit for carrying out the above-described method, said kit comprising:
i) means for detecting a compound, optionally labelled compound of the invention, and,
ii) optionally, buffering agents, and other labelling reagents and instructions for use of the kit.

The above methods may be advantageously used for monitoring human patients suffering from disorders selected from the group consisting of infectious, autoimmune and inflammatory disorders, characterized by neutrophilic inflammation, where PR3 is used a biomarker of the progression or the state of the disease, including without limitation cystic fibrosis, COPD, acute lung injury, granulomatosis with polyangiitis, rheumatoid arthritis, and, cancers associated with neutrophil recruitment, such as lung cancer and bladder cancer.

### Synthesis of the Selective Proteinase 3 Inhibitors of the Invention

The synthesis of phosphonic analogue of alanine started with the preparation of tri(4-chlorophenyl)phosphite from 4-chlorophenol and phosphorus trichloride, that may be performed as described previously (15) and summarized in the scheme below:

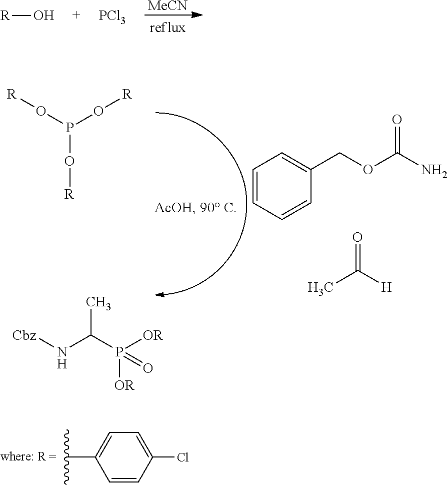

Briefly, to the solution of 4-chlorophenol (30 mmol) in acetonitrile (50 ml) phosphorus trichloride (10 mmol) was added. The reaction mixture was refluxed for 6 h and the volatile elements were removed under reduced pressure. The resulting crude phosphite obtained as yellow oil was used directly for an amidoalkylation reaction with benzyl carbamate (12 mmol) and acetaldehyde (12 mmol). The reaction was performed in refluxing acetic acid for 3 h according to Oleksyszyn's method yielding Cbz-Ala^{P}(OC₆H₄-4-Cl)₂. Next, the Cbz-protecting group was removed with 33% hydrobromic acid solution in acetic acid (2 h, r.t.). The volatile components were removed under reduced pressure and the product was crystallized from methanol/diethyl ether giving HBr*H₂N-Ala^{P}(O—C₆H₄-4-Cl)₂ as white solid.

The synthesis of phosphonic analogue of norvaline was done similarly to the analogue of alanine and started with the preparation of tri(4-chlorophenyl)phosphite from 4-chlorophenol and phosphorus trichloride. To the solution of 4-chlorophenol (30 mmol) in acetonitrile (50 ml) phosphorus trichloride (10 mmol) was added. The reaction mixture was refluxed for 6 h and the volatile elements were removed under reduced pressure. The resulting crude phosphite obtained as yellow oil was used directly for an amidoalkylation reaction with benzyl carbamate (12 mmol) and n-butyl aldehyde (12 mmol). The reaction was performed in refluxing acetic acid for 3 h according to Oleksyszyn's method yielding Cbz-Avl^{P}(OC₆H₄-4-Cl)₂. Next, the Cbz-protecting group was removed with 33% hydrobromic acid solution in acetic acid (2 h, r.t.). The volatile components were removed under reduced pressure and the product was crystallized from methanol/diethyl ether giving HBr*H₂N-Avl^{P}(O—C₆H₄-4-Cl)₂ as white solid.

All the peptides can be synthesized manually following conventional peptide synthesis methods, such as the solid-phase method using Fmoc chemistry. The following amino acid derivatives were used: Fmoc-Pro, Fmoc-Tyr(tBu), Fmoc-Asp(OtBu), Fmoc-Trp(Boc), Fmoc-Ile, Fmoc-Val. The protected derivative of C-terminal amino acid residue, Fmoc-Asp(OtBu), is for example attached to the 2-chlorotritylchloride resin (substitution of CI 1.46 mequiv/g) (Calbiochem-Novabiochem AG, Switzerland) in the presence of an equimolar amount of DIPEA (based on the amino acid) under anhydrous conditions in DCM solution. Peptide chains are elongated in consecutive cycles of deprotection and coupling. Deprotection is performed with 20% piperidine in DMF/NMP (1:1, v/v) with the addition of 1% Triton X-100, whereas chain elongation was achieved with standard DIC/HOBt chemistry; 3 equiv of protected amino acid derivatives were used. The same method may be applied for coupling biotine or fluorescent compounds (used as N-protected Fmoc derivatives). For N-terminal acetyl derivative the 10 fold molar excess of N-acetylaimidasole in DMF is used. After completing the syntheses, the peptides are cleaved from the resin with TFE:hexan:acetic acid mixture.

Fully protected peptides are dissolved in DMF and its carboxyl group is activated using DIPCI to such mixture the solution of HBr×H₂N-Ala^{P}(O—C₆H₄-4-Cl)₂ in DMF and DIPEA is added. The mixture is stirred for 6 hours and after that DMF is evaporated under reduced pressure. The obtained compound are resuspend in TFA/phenol/triisopropylsilane/H₂O (88:5:2:5, v/v/v/v) mixture for 2 hours in order to removed side chain protection groups.

The crude peptides are purified using HPLC on a Beckman Gold System (Beckman, USA) with an RP Kromasil-100, C8, 5 μm column (8×250 mm) (Knauer, Germany).

The details of the preparation of several exemplary embodiments of the disclosed PR3 selective inhibitors are described below.

### LEGENDS OF THE FIGURES

FIG. 1: Overall structure of the peptide diphenyl phosphonate inhibitors as illustrated with Compound 1 (Ac-Asp-Tyr-Asp-Ala^{P}(O—C₆H₄-4-Cl)₂ (A) and selective inhibition of Proteinase 3 by compound 1 (B)

FIG. 2: Inhibition of PR3 by increasing amounts of compound 2 (Ac-Pro-Tyr-Asp-Ala^{P}(O—C₆H₄-4-Cl)₂(A), and selectivity towards Proteinase 3 (B)

FIG. 3: Inhibition of wild type PR3 and two molecular variants (PR31217R and PR3K99L) by increasing amounts of compound 2

FIG. 4: Visualization by Western Blotting analysis and streptavidin-peroxydase staining of the selective interaction of biotinylated compound 2 (=compound 4) with purified proteinase 3 (A) a neutrophil lysate at pH7.4 (B) and a neutrophil lysate at pH 5 to 9 (C)

### EXAMPLES

### Materials and Methods

### Chemicals

FRET substrates and p-nitroanilide substrates were synthesized by Genecust (Dudelange Luxembourg)

### Production of Recombinant PR3 Mutants

Recombinant PR3K99L and PR31217R were produced in Drosophila S2 cells using the calcium phosphate method, and stable cell lines were selected using puromycin. Proteins were purified by affinity chromatography on Chelating Sepharose and analyzed by SDS-PAGE. The total protein concentration was determined with a BCA Protein Assay

### General Method of Synthesis of Peptidyl-Phosphonate Inhibitors

The synthesis of phosphonic analogue of alanine started with the preparation of tri(4-chlorophenyl)phosphite from 4-chlorophenol and phosphorus trichloride as described previously (15). Briefly, 4-chlorophenol (30 mmol) was dissolved in acetonitrile (50 ml) followed by the addition of phosphorus trichloride (10 mmol). The reaction mixture was refluxed for 6 h and the volatile elements were removed in vacuum. The resulting crude phosphite obtained as yellow oil was used directly for an amidoalkylation reaction with benzyl carbamate (12 mmol) and acetaldehyde (12 mmol). The reaction was performed in refluxing acetic acid for 3 h according to Oleksyszyn's method (16) yielding Cbz-Ala^{P}(O—C₆H₄-4-Cl)₂ as a white solid: 56%; HRMS: calcd for (C₂₂H₂₀Cl₂NO₅P)H⁺, 480.0534. found, 480.0533. ¹H NMR (CDCl₃, ppm): δ 7.43-6.97 (m, 14H), 5.22-5.08 (m, 2H), 4.74-4.37 (m, 1H), 1.56 (dd, J=18.2, 7.4 Hz, 3H); ³¹P NMR (CDCl₃, ppm): δ 19.56 (s). Next, the Cbz-protecting group was removed with 33% hydrobromic acid solution in acetic acid (2 h, r.t.). The volatile components were removed under reduced pressure and the product was crystallized from methanol/diethyl ether giving HBr*H₂N-Ala^{P}(O—C₆H₄-4-Cl)₂ as white solid: 97%; HRMS: calcd for (C₁₄H₁₄Cl₂NO₃P)H⁺, 346.0167. found, 346.0172. ¹H NMR (DMSO-d₆, ppm): δ 8.85 (s, 3H), 7.57-7.44 (m, 4H), 7.36-7.16 (m, 4H), 4.45-4.24 (m, 1H), 1.55 (dd, J=18.3, 7.2 Hz, 3H); ³¹P NMR (CDCl₃, ppm): δ 16.49 (s).

The synthesis of phosphonic analogue of norvaline was done similarly to the analoge of alanine and started with the preparation of tri(4-chlorophenyl)phosphite from 4-chlorophenol and phosphorus trichloride. To the solution of 4-chlorophenol (30 mmol) in acetonitrile (50 ml) phosphorus trichloride (10 mmol) was added. The reaction mixture was refluxed for 6 h and the volatile elements were removed under reduced pressure. The resulting crude phosphite obtained as yellow oil was used directly for an amidoalkylation reaction with benzyl carbamate (12 mmol) and n-butyl aldehyde (12 mmol). The reaction was performed in refluxing acetic acid for 3 h according to Oleksyszyn's method yielding Cbz-Avl^{P}(OC₆H₄-4-Cl)₂. Next, the Cbz-protecting group was removed with 33% hydrobromic acid solution in acetic acid (2 h, r.t.). The volatile components were removed under reduced pressure and the product was crystallized from methanol/diethyl ether giving HBr*H₂N-Avl^{P}(O—C₆H₄-4-Cl)₂ as white solid.

All the peptides were synthesized manually following the solid-phase method using Fmoc chemistry. The following amino acid derivatives were used: Fmoc-Pro, Fmoc-Tyr(tBu), Fmoc-Asp(OtBu), Fmoc-Trp(Boc), Fmoc-Ile, Fmoc-Val. The protected derivative of C-terminal amino acid residue, Fmoc-Asp(OtBu), was attached to the 2-chlorotritylchloride resin (substitution of Cl 1.46 mequiv/g) (Calbiochem-Novabiochem AG, Switzerland) in the presence of an equimolar amount of DIPEA (based on the amino acid) under anhydrous conditions in DCM solution. Peptide chains were elongated in consecutive cycles of deprotection and coupling. Deprotection was performed with 20% piperidine in DMF/NMP (1:1, v/v) with the addition of 1% Triton X-100, whereas chain elongation was achieved with standard DIC/HOBt chemistry; 3 equiv of protected amino acid derivatives were used. The same method was applied for coupling biotin and fluorescent compounds (used as N-protected Fmoc derivatives). For N-terminal acetyl derivative the 10 fold molar excess of N-acetylaimidasole in DMF was used. After completing the syntheses, the peptides were cleaved from the resin with TFE:DCM:acetic acid (2:6:2, v/v/v/) mixture.

Fully protected peptides were dissolved in DMF and its carboxyl group was activated using DIPCI to such mixture the solution of HBr×H₂N-Ala^{P}(O—C₆H₄-4-Cl)₂ in DMF and DIPEA was added. The mixture was stirred for 6 hours and after that DMF was evaporated under reduced pressure. The obtained compound was resuspended in TFA/phenol/triisopropylsilane/H₂O (88:5:2:5, v/v/v/v) mixture for 2 hours in order to removed side chain protection groups.

The crude peptides were purified using HPLC on a Beckman Gold System (Beckman, USA) with an RP Kromasil-100, C8, 5 μm column (8×250 mm) (Knauer, Germany). The solvent systems were 0.1% TFA (A) and 80% acetonitrile in A (B). Either isocratic conditions or a linear gradient were applied (flow rate 3.0 mL/min, monitored at 226 nm). The purity of the synthesized peptides was checked on an RP Kromasil 100, C8, 5 μm column (4.6×250 mm) (Knauer, Germany). The solvent system was as above. Linear gradient from 10% to 90% B for 30 min, flow rate 1 mL/min, monitored at 226 nm. The mass spectrometry analysis was carried out on a MALDI MS (a Biflex III MALDI-TOF spectrometer, Bruker Daltonics, Germany) using a-CCA matrix.

### Protease Inhibition Assays

The inactivation of proteases by substrate analog inhibitors in the presence of the substrate by competition for the enzyme-binding site, was measured by the method of Tian and Tsou (17). In this system, product formation in the presence of an irreversible inhibitor approaches an asymptote, as described by

log([P∞]−[P])=log [P∞]−0.43A[Y]t

where [P∞] is the concentration of product formed at time approaching infinity, [P] is the concentration of product at time t, [Y] is the inhibitor concentration, and A is the apparent inhibition rate constant in the presence of the substrate. A is given by

A=k₊₀/(1+K⁻¹[S])

where k₊₀ is the rate constant for association of the inhibitor with the enzyme, K⁻¹ is the inverted Michaelis constant, and [S] the substrate concentration. The apparent inhibition rate constant A is the slope of a plot of log([P∞]−[P]) against t, to give the second-order rate constant of inhibition k₊₀

### Example 1

This example describes the synthesis of compound 1 Ac-Asp-Tyr-Asp-Ala^{P}(O—C₆H₄-4-Cl)₂ Ac-Asp-Tyr-Asp-Ala^{P}(O—C₆H₄-4-Cl)₂ was synthesized manually following the solid-phase method using Fmoc chemistry. The following amino acid derivatives were used: Fmoc-Tyr(tBu), Fmoc-Asp(OtBu). The protected derivative of C-terminal amino acid residue, Fmoc-Asp(OtBu) 1.46 mmol, was attached to the 0.25 g 2-chlorotritylchloride resin (substitution of Cl 1.46 mequiv/g) (Calbiochem-Novabiochem AG, Switzerland) in the presence of an equimolar amount of DIPEA (based on the amino acid) under anhydrous conditions in 5 ml of DCM solution. The final effective substitution was determined spectrophotometrically and was equal to 0.82 mequiv/g). Peptide chains were elongated in consecutive cycles of deprotection and coupling. Deprotection was performed with 5 ml of 20% piperidine in DMF/NMP (1:1, v/v) with the addition of 1% Triton X-100, whereas chain elongation was achieved with standard DIC/HOBt chemistry; 0.36 mmol of each protected amino acid derivatives; Fmoc-Tyr(tBu) followed by Fmoc-Asp(OtBu) were used. The introduction of N-terminal acetyl group was obtained by using 10 fold molar (3.6 mmol) excess of N-acetylimidazole in 2 ml DMF and whole mixture was stirred for 1 hour. The procedure was repeated 3 times after completing the syntheses, the peptides were cleaved from the resin with 10 ml of TFE:DCM:acetic acid mixture (2:6:2, v/v/v).

0.3 mmoles of fully protected peptide was dissolved in 2 ml DMF and its carboxyl group was activated using equimolar amount of DIPCI. To such mixture the 0.5 mL of 0.31 mmoles of HBr×H₂N-Ala^{P}(O—C₆H₄-4-Cl)₂ in DMF and 0.31 mmol of DIPEA was added. The mixture was stirred for 6 hours and after that DMF was evaporated under reduced pressure. The obtained compound was suspended in 4 ml of TFA/phenol/triisopropylsilane/H₂O (88:5:2:5, v/v/v/v) mixture for 2 hours in order to removed side chain protection groups. The crude peptide was precipitated with anhydrous diethyl ether and centrifuged at 15000 g (centrifuge AK-30, Sigma, Germany). The white peptide pallet was dissolved in 10 ml of distilled water and freeze dried.

The crude peptides were purified using HPLC on a Beckman Gold System (Beckman, USA) with an RP Kromasil-100, C8, 5 μm column (8×250 mm) (Knauer, Germany). The solvent systems were 0.1% TFA (A) and 80% acetonitrile in A (B). Either isocratic conditions or a linear gradient were applied (flow rate 3.0 mL/min, monitored at 226 nm). The purity of the synthesized peptides was checked on an RP Kromasil 100, C8, 5 μm column (4.6×250 mm) (Knauer, Germany). The solvent system was as above. Linear gradient from 10% to 90% B for 30 min, flow rate 1 mL/min, monitored at 226 nm. The mass spectrometry analysis was carried out on a MALDI MS (a Biflex III MALDI-TOF spectrometer, Bruker Daltonics, Germany) using a-CCA matrix.

HRMS: calcd for (MH⁺), 781.5325. found, 781.5586.

HNMR ¹H NMR (DMSO-d₆, ppm): δ 11.03 (s 2H), 8.05 (s, 4H), 7.37-7.12 (m, 12H), 5.37 (s, 1H), 4.92-4.24 (m, 4H), 3.44-2.66 (m, 6H), 1.84-1.47 (m, 4H)

### Example 2

This example describes the synthesis of compound 2 Ac-Pro-Tyr-Asp-Ala^{P}(O—C₆H₄-4-Cl)₂ Ac-Pro-Tyr-Asp-Ala^{P}(O—C₆H₄-4-Cl)₂ was synthesized manually following the solid-phase method using Fmoc chemistry. The following amino acid derivatives were used: Fmoc-Pro, Fmoc-Tyr(tBu), Fmoc-Asp(OtBu). The protected derivative of C-terminal amino acid residue, Fmoc-Asp(OtBu) 1.46 mmol, was attached to the 0.25 g 2-chlorotritylchloride resin (substitution of Cl 1.46 mequiv/g) (Calbiochem-Novabiochem AG, Switzerland) in the presence of an equimolar amount of DIPEA (based on the amino acid) under anhydrous conditions in 5 ml of DCM solution. The final effective substitution was determined spectrophotometrically and was equal to 0.82 mequiv/g). Peptide chains were elongated in consecutive cycles of deprotection and coupling. Deprotection was performed with 5 ml of 20% piperidine in DMF/NMP (1:1, v/v) with the addition of 1% Triton X-100, whereas chain elongation was achieved with standard DIC/HOBt chemistry; 0.36 mmol of each protected amino acid derivatives; Fmoc-Tyr(tBu) followed by Fmoc-Pro were used. The introduction of N-terminal acetyl group was obtained by using 10 fold molar (3.6 mmol) excess of N-acetylimidazole in 2 ml DMF and whole mixture was stirred for 1 hour. The procedure was repeated 3 times. After completing the syntheses, the peptides were cleaved from the resin with 10 ml of TFE:DCM:acetic acid mixture (2:6:2, v/v/v).

0.3 mmoles of fully protected peptide was dissolved in 2 ml DMF and its carboxyl group was activated using equimolar amount of DIPCI. To such mixture the 0.5 mL of 0.31 mmoles of HBr×H₂N-Ala^{P}(O—C₆H₄-4-Cl)₂ in DMF and 0.31 mmol of DIPEA was added. The mixture was stirred for 6 hours and after that DMF was evaporated under reduced pressure. The obtained compound was suspended in 4 ml of TFA/phenol/triisopropylsilane/H₂O (88:5:2:5, v/v/v/v) mixture for 2 hours in order to removed side chain protection groups. The crude peptide was precipitated with anhydrous diethyl ether and centrifuged at 15000 g (centrifuge AK-30, Sigma, Germany). The white peptide pallet was dissolved in 10 ml of distilled water and freeze dried.

The crude peptides were purified using HPLC on a Beckman Gold System (Beckman, USA) with an RP Kromasil-100, C8, 5 μm column (8×250 mm) (Knauer, Germany). The solvent systems were 0.1% TFA (A) and 80% acetonitrile in A (B). Either isocratic conditions or a linear gradient were applied (flow rate 3.0 mL/min, monitored at 226 nm). The purity of the synthesized peptides was checked on an RP Kromasil 100, C8, 5 μm column (4.6×250 mm) (Knauer, Germany). The solvent system was as above. Linear gradient from 10% to 90% B for 30 min, flow rate 1 mL/min, monitored at 226 nm. The mass spectrometry analysis was carried out on a MALDI MS (a Biflex III MALDI-TOF spectrometer, Bruker Daltonics, Germany) using a-CCA matrix.

HRMS: calcd for (MH⁺), 763.5684. found, 763.5712.

HNMR ¹H NMR (DMSO-d₆, ppm): δ 11.03 (s 1H), 8.08 (s, 3H), 7.37-7.12 (m, 12H), 5.27 (s, 1H), 4.92-4.37 (m, 4H), 3.44-2.66 (m, 4H), 2.01 (m, 6H) 1.84-1.47 (m, 4H)

### Example 3

This example describes the synthesis of compound 3 Biotin-Asp-Tyr-Asp-Ala^{P}(O—C₆H₄-4-Cl)₂

Biotin-Asp-Tyr-Asp-Ala^{P}(O—C₆H₄-4-Cl)₂ was synthesized manually following the solid-phase method using Fmoc chemistry. The following amino acid derivatives were used: Fmoc-Tyr(tBu), Fmoc-Asp(OtBu)-OH. The protected derivative of C-terminal amino acid residue, Fmoc-Asp(OtBu) 1.46 mmol, was attached to the 0.25 g 2-chlorotritylchloride resin (substitution of Cl 1.46 mequiv/g) (Calbiochem-Novabiochem AG, Switzerland) in the presence of an equimolar amount of DIPEA (based on the amino acid) under anhydrous conditions in 5 ml of DCM solution. The final effective substitution was determined spectrophotometrically and was equal to 0.82 mequiv/g). Peptide chains were elongated in consecutive cycles of deprotection and coupling. Deprotection was performed with 5 ml of 20% piperidine in DMF/NMP (1:1, v/v) with the addition of 1% Triton X-100, whereas chain elongation was achieved with standard DIC/HOBt chemistry; 0.36 mmol of each protected amino acid derivatives; Fmoc-Tyr(tBu) followed by and Fmoc-Asp(OtBu)) were used. The introduction of N-terminal biotin group was obtained by using 5 fold molar (1.8 mmol) excess of biotin and DIPCI in 2 ml anhydrous DMSO and whole mixture was stirred for 6 hour at 30 C The whole procedure was repeated 2 times. After completing the syntheses, the peptides were cleaved from the resin with 10 ml of TFE:DCM:acetic acid mixture (2:6:2, v/v/v).

0.3 mmoles of fully protected peptide was dissolved in 2 ml DMF and its carboxyl group was activated using equimolar amount of DIPCI. To such mixture the 0.5 mL of 0.31 mmoles of HBr×H₂N-Ala^{P}(O—C₆H₄-4-Cl)₂ in DMF and 0.31 mmol of DIPEA was added. The mixture was stirred for 6 hours and after that DMF was evaporated under reduced pressure. The obtained compound was suspended in 4 ml of TFA/phenol/triisopropylsilane/H₂O (88:5:2:5, v/v/v/v) mixture for 2 hours in order to removed side chain protection groups. The crude peptide was precipitated with anhydrous diethyl ether and centrifuged at 15000 g (centrifuge AK-30, Sigma, Germany). The white peptide pallet was dissolved in 10 ml of distilled water and freeze dried.

The crude peptides were purified using HPLC on a Beckman Gold System (Beckman, USA) with an RP Kromasil-100, C8, 5 μm column (8×250 mm) (Knauer, Germany). The solvent systems were 0.1% TFA (A) and 80% acetonitrile in A (B). Either isocratic conditions or a linear gradient were applied (flow rate 3.0 mL/min, monitored at 226 nm). The purity of the synthesized peptides was checked on an RP Kromasil 100, C8, 5 μm column (4.6×250 mm) (Knauer, Germany). The solvent system was as above. Linear gradient from 10% to 90% B for 30 min, flow rate 1 mL/min, monitored at 226 nm. The mass spectrometry analysis was carried out on a MALDI MS (a Biflex III MALDI-TOF spectrometer, Bruker Daltonics, Germany) using a-CCA matrix.

HRMS: calcd for (MH⁺), 965.7991. found, 965.8672.

HNMR ¹H NMR (DMSO-d₆, ppm): δ 11.03 (s 2H), 8.05 (s, 4H), 7.37-7.12 (m, 12H), 6.01 (s, 2H) 5.37 (s, 1H), 4.92-4.24 (m, 4H), 3.44-2.66 (m, 8H), 1.84-1.47 (m, 8H)

### Example 4

This example describes the synthesis of compound 4 Biotin-Pro-Tyr-Asp-Ala^{P}(O—C₆H₄-4-Cl)₂.

Biotin-Pro-Tyr-Asp-Ala^{P}(O—C₆H₄-4-Cl)₂ was synthesized manually following the solid-phase method using Fmoc chemistry. The following amino acid derivatives were used: Fmoc-Pro, Fmoc-Tyr(tBu), Fmoc-Asp(OtBu). The protected derivative of C-terminal amino acid residue, Fmoc-Asp(OtBu) 1.46 mmol, was attached to the 0.25 g 2-chlorotritylchloride resin (substitution of Cl 1.46 mequiv/g) (Calbiochem-Novabiochem AG, Switzerland) in the presence of an equimolar amount of DIPEA (based on the amino acid) under anhydrous conditions in 5 ml of DCM solution. The final effective substitution was determined spectrophotometrically and was equal to 0.82 mequiv/g). Peptide chains were elongated in consecutive cycles of deprotection and coupling. Deprotection was performed with 5 ml of 20% piperidine in DMF/NMP (1:1, v/v) with the addition of 1% Triton X-100, whereas chain elongation was achieved with standard DIC/HOBt chemistry; 0.36 mmol of each protected amino acid derivatives; Fmoc-Tyr(tBu) followed by Fmoc-Pro were used. The introduction of N-terminal biotin group was obtained by using 5 fold molar (1.8 mmol) excess of biotin and DIPCI in 2 ml anhydrous DMSO and whole mixture was stirred for 6 hour at 30 C. The whole procedure was repeated 2 times. After completing the syntheses, the peptides were cleaved from the resin with 10 ml of TFE:DCM:acetic acid mixture (2:6:2, v/v/v).

0.3 mmoles of fully protected peptide was dissolved in 2 ml DMF and its carboxyl group was activated using equimolar amount of DIPCI. To such mixture the 0.5 mL of 0.31 mmoles of HBr×H₂N-Ala^{P}(O—C₆H₄-4-Cl)₂ in DMF and 0.31 mmol of DIPEA was added. The mixture was stirred for 6 hours and after that DMF was evaporated under reduced pressure. The obtained compound was suspended in 4 ml of TFA/phenol/triisopropylsilane/H₂O (88:5:2:5, v/v/v/v) mixture for 2 hours in order to removed side chain protection groups. The crude peptide was precipitated with anhydrous diethyl ether and centrifuged at 15000 g (centrifuge AK-30, Sigma, Germany). The white peptide pallet was dissolved in 10 ml of distilled water and freeze dried.

The crude peptides were purified using HPLC on a Beckman Gold System (Beckman, USA) with an RP Kromasil-100, C8, 5 μm column (8×250 mm) (Knauer, Germany). The solvent systems were 0.1% TFA (A) and 80% acetonitrile in A (B). Either isocratic conditions or a linear gradient were applied (flow rate 3.0 mL/min, monitored at 226 nm). The purity of the synthesized peptides was checked on an RP Kromasil 100, C8, 5 μm column (4.6×250 mm) (Knauer, Germany). The solvent system was as above. Linear gradient from 10% to 90% B for 30 min, flow rate 1 mL/min, monitored at 226 nm. The mass spectrometry analysis was carried out on a MALDI MS (a Biflex III MALDI-TOF spectrometer, Bruker Daltonics, Germany) using a-CCA matrix.

HRMS: calcd for (MH⁺), 947.8269. found, 947.8472.

HNMR ¹H NMR (DMSO-d₆, ppm): δ 11.03 (s 1H), 8.08 (s, 3H), 7.37-7.12 (m, 12H), 5.98 (s, 2H) 5.27 (s, 1H), 4.92-4.37 (m, 4H), 3.44-2.66 (m, 4H), 2.01-2.12 (m, 8H) 1.84-1.47 (m, 6H)

### Example 5

This example describes the synthesis of compound 5 Biotin-Pro-Tyr-Asp-Avl^{P}(O—C₆H₄-4-Cl)₂.

Biotin-Pro-Tyr-Asp-Ala^{P}(O—C₆H₄-4-Cl)₂ was synthesized manually following the solid-phase method using Fmoc chemistry. The following amino acid derivatives were used: Fmoc-Pro, Fmoc-Tyr(tBu), Fmoc-Asp(OtBu). The protected derivative of C-terminal amino acid residue, Fmoc-Asp(OtBu)) 1.46 mmol, was attached to the 0.25 g 2-chlorotritylchloride resin (substitution of Cl 1.46 mequiv/g) (Calbiochem-Novabiochem AG, Switzerland) in the presence of an equimolar amount of DIPEA (based on the amino acid) under anhydrous conditions in 5 ml of DCM solution. The final effective substitution was determined spectrophotometrically and was equal to 0.82 mequiv/g). Peptide chains were elongated in consecutive cycles of deprotection and coupling. Deprotection was performed with 5 ml of 20% piperidine in DMF/NMP (1:1, v/v) with the addition of 1% Triton X-100, whereas chain elongation was achieved with standard DIC/HOBt chemistry; 0.36 mmol of each protected amino acid derivatives; Fmoc-Tyr(tBu) followed by Fmoc-Pro were used. The introduction of N-terminal biotin group was obtained by using 5 fold molar (1.8 mmol) excess of biotin and DIPCI in 2 ml anhydrous DMSO and whole mixture was stirred for 6 hour at 30 C. The whole procedure was repeated 2 times. After completing the syntheses, the peptides were cleaved from the resin with 10 ml of TFE:DCM:acetic acid mixture (2:6:2, v/v/v).

0.3 mmoles of fully protected peptide was dissolved in 2 ml DMF and its carboxyl group was activated using equimolar amount of DIPCI. To such mixture the 0.5 mL of 0.31 mmoles of HBr×H₂N-Avl^{P}(O—C₆H₄-4-Cl)₂ in DMF and 0.31 mmol of DIPEA was added. The mixture was stirred for 6 hours and after that DMF was evaporated under reduced pressure. The obtained compound was suspended in 4 ml of TFA/phenol/triisopropylsilane/H₂O (88:5:2:5, v/v/v/v) mixture for 2 hours in order to removed side chain protection groups. The crude peptide was precipitated with anhydrous diethyl ether and centrifuged at 15000 g (centrifuge AK-30, Sigma, Germany). The white peptide pallet was dissolved in 10 ml of distilled water and freeze dried. The crude peptides were purified using HPLC on a Beckman Gold System (Beckman, USA) with an RP Kromasil-100, C8, 5 μm column (8×250 mm) (Knauer, Germany). The solvent systems were 0.1% TFA (A) and 80% acetonitrile in A (B). Either isocratic conditions or a linear gradient were applied (flow rate 3.0 mL/min, monitored at 226 nm). The purity of the synthesized peptides was checked on an RP Kromasil 100, C8, 5 μm column (4.6×250 mm) (Knauer, Germany). The solvent system was as above. Linear gradient from 10% to 90% B for 30 min, flow rate 1 mL/min, monitored at 226 nm. The mass spectrometry analysis was carried out on a MALDI MS (a Biflex III MALDI-TOF spectrometer, Bruker Daltonics, Germany) using a-CCA matrix.

### Example 6

This example describes the synthesis of compound 6 Biotin-Val-Tyr-Asp-Ala^{P}(O—C₆H₄-4-Cl)₂.

Biotin-Val-Tyr-Asp-Ala^{P}(O—C₆H₄-4-Cl)₂ was synthesized manually following the solid-phase method using Fmoc chemistry. The following amino acid derivatives were used: Fmoc-Val, Fmoc-Tyr(tBu), Fmoc-Asp(OtBu). The protected derivative of C-terminal amino acid residue, Fmoc-Asp(OtBu) 1.46 mmol, was attached to the 0.25 g 2-chlorotritylchloride resin (substitution of Cl 1.46 mequiv/g) (Calbiochem-Novabiochem AG, Switzerland) in the presence of an equimolar amount of DIPEA (based on the amino acid) under anhydrous conditions in 5 ml of DCM solution. The final effective substitution was determined spectrophotometrically and was equal to 0.82 mequiv/g). Peptide chains were elongated in consecutive cycles of deprotection and coupling. Deprotection was performed with 5 ml of 20% piperidine in DMF/NMP (1:1, v/v) with the addition of 1% Triton X-100, whereas chain elongation was achieved with standard DIC/HOBt chemistry; 0.36 mmol of each protected amino acid derivatives; Fmoc-Tyr followed by Fmoc-Val were used. After completing the syntheses, the peptides were cleaved from the resin with 10 ml of TFE:DCM:acetic acid mixture (2:6:2, v/v/v).

0.3 mmoles of fully protected peptide was dissolved in 2 ml DMF and its carboxyl group was activated using equimolar amount of DIPCI. To such mixture the 0.5 mL of 0.31 mmoles of HBr×H₂N-Ala^{P}(O—C₆H₄-4-Cl)₂ in DMF and 0.31 mmol of DIPEA was added. The mixture was stirred for 6 hours and after that DMF was evaporated under reduced pressure. The obtained compound was suspended in 4 ml of TFA/phenol/triisopropylsilane/H₂O (88:5:2:5, v/v/v/v) mixture for 2 hours in order to removed side chain protection groups. The crude peptide was precipitated with anhydrous diethyl ether and centrifuged at 15000 g (centrifuge AK-30, Sigma, Germany).

The white peptide pallet was dissolved in 10 ml of distilled water and freeze dried. The crude peptides were purified using HPLC on a Beckman Gold System (Beckman, USA) with an RP Kromasil-100, C8, 5 μm column (8×250 mm) (Knauer, Germany). The solvent systems were 0.1% TFA (A) and 80% acetonitrile in A (B). Either isocratic conditions or a linear gradient were applied (flow rate 3.0 mL/min, monitored at 226 nm). The purity of the synthesized peptides was checked on an RP Kromasil 100, C8, 5 μm column (4.6×250 mm) (Knauer, Germany). The solvent system was as above. Linear gradient from 10% to 90% B for 30 min, flow rate 1 mL/min, monitored at 226 nm. The mass spectrometry analysis was carried out on a MALDI MS (a Biflex III MALDI-TOF spectrometer, Bruker Daltonics, Germany) using a-CCA matrix.

### Example 7

This example describes the synthesis of compound 7 Ac-Pro-Tyr-Phe-Ala^{P}(O—C₆H₄-4-Cl)₂. Ac-Pro-Tyr-Phe-Ala^{P}(O—C₆H₄-4-Cl)₂ was synthesized manually following the solid-phase method using Fmoc chemistry. The following amino acid derivatives were used: Fmoc-Pro, Fmoc-Tyr(tBu), Fmoc-Phe The protected derivative of C-terminal amino acid residue, Fmoc-Phe) 1.46 mmol, was attached to the 0.25 g 2-chlorotritylchloride resin (substitution of Cl 1.46 mequiv/g) (Calbiochem-Novabiochem AG, Switzerland) in the presence of an equimolar amount of DIPEA (based on the amino acid) under anhydrous conditions in 5 ml of DCM solution. The final effective substitution was determined spectrophotometrically and was equal to 0.82 mequiv/g). Peptide chains were elongated in consecutive cycles of deprotection and coupling. Deprotection was performed with 5 ml of 20% piperidine in DMF/NMP (1:1, v/v) with the addition of 1% Triton X-100, whereas chain elongation was achieved with standard DIC/HOBt chemistry; 0.36 mmol of each protected amino acid derivatives; followed by Ac-Pro were used. After completing the syntheses, the peptides were cleaved from the resin with 10 ml of TFE:DCM:acetic acid mixture (2:6:2, v/v/v).

0.3 mmoles of fully protected peptide was dissolved in 2 ml DMF and its carboxyl group was activated using equimolar amount of DIPCI. To such mixture the 0.5 mL of 0.31 mmoles of HBr×H₂N-Ala^{P}(O—C₆H₄-4-Cl)₂ in DMF and 0.31 mmol of DIPEA was added. The mixture was stirred for 6 hours and after that DMF was evaporated under reduced pressure. The obtained compound was suspended in 4 ml of TFA/phenol/triisopropylsilane/H₂O (88:5:2:5, v/v/v/v) mixture for 2 hours in order to removed side chain protection groups. The crude peptide was precipitated with anhydrous diethyl ether and centrifuged at 15000 g (centrifuge AK-30, Sigma, Germany). The white peptide pallet was dissolved in 10 ml of distilled water and freeze dried.

The crude peptides were purified using HPLC on a Beckman Gold System (Beckman, USA) with an RP Kromasil-100, C8, 5 μm column (8×250 mm) (Knauer, Germany). The solvent systems were 0.1% TFA (A) and 80% acetonitrile in A (B). Either isocratic conditions or a linear gradient were applied (flow rate 3.0 mL/min, monitored at 226 nm). The purity of the synthesized peptides was checked on an RP Kromasil 100, C8, 5 μm column (4.6×250 mm) (Knauer, Germany). The solvent system was as above. Linear gradient from 10% to 90% B for 30 min, flow rate 1 mL/min, monitored at 226 nm. The mass spectrometry analysis was carried out on a MALDI MS (a Biflex III MALDI-TOF spectrometer, Bruker Daltonics, Germany) using a-CCA matrix.

HRMS: calcd for (MH+), 795.5342. found, 795.6821.

HNMR 1H NMR (DMSO-d6, ppm): 8.10 (s, 3H), 7.42-6.74 (m, 17H), 5.31 (s, 1H), 4.92-4.29 (m, 3H), 3.44-2.66 (m, 7H), 2.01-2.12 (m, 8H) 1.34-1.18 (m, 3H)

### Example 8

This example describes the synthesis of compound 8 CH₃—(CH₂)₄—Pro-Tyr-Asp-Ala^{P}(O—C₆H₄-4-Cl)₂.

CH₃—(CH₂)₄—Pro-Tyr-Asp-Ala^{P}(O—C₆H₄-4-Cl)₂ was synthesized manually following the solid-phase method using Fmoc chemistry. The following amino acid derivatives were used: Fmoc-Pro, Fmoc-Tyr(tBu), Fmoc-Asp(OtBu). The protected derivative of C-terminal amino acid residue, Fmoc-Asp(OtBu) 1.46 mmol, was attached to the 0.25 g 2-chlorotritylchloride resin (substitution of Cl 1.46 mequiv/g) (Calbiochem-Novabiochem AG, Switzerland) in the presence of an equimolar amount of DIPEA (based on the amino acid) under anhydrous conditions in 5 ml of DCM solution. The final effective substitution was determined spectrophotometrically and was equal to 0.82 mequiv/g). Peptide chains were elongated in consecutive cycles of deprotection and coupling. Deprotection was performed with 5 ml of 20% piperidine in DMF/NMP (1:1, v/v) with the addition of 1% Triton X-100, whereas chain elongation was achieved with standard DIC/HOBt chemistry; 0.36 mmol of each protected amino acid derivatives; Fmoc-Tyr(tBu) followed by Fmoc-Pro were used. The introduction of N-terminal valeric acid moiety was obtained by using 5 fold molar (1.8 mmol) excess of valeric acid and DIPCI in 2 ml DMF and whole mixture was stirred for 2 hours at 30 C. The whole procedure was repeated 2 times. After completing the syntheses, the peptides were cleaved from the resin with 10 ml of TFE:DCM:acetic acid mixture (2:6:2, v/v/v).

0.3 mmoles of fully protected peptide was dissolved in 2 ml DMF and its carboxyl group was activated using equimolar amount of DIPCI. To such mixture the 0.5 mL of 0.31 mmoles of HBr×H₂N-Ala^{P}(O—C₆H₄-4-Cl)₂ in DMF and 0.31 mmol of DIPEA was added. The mixture was stirred for 6 hours and after that DMF was evaporated under reduced pressure. The obtained compound was suspended in 4 ml of TFA/phenol/triisopropylsilane/H₂O (88:5:2:5, v/v/v/v) mixture for 2 hours in order to removed side chain protection groups. The crude peptide was precipitated with anhydrous diethyl ether and centrifuged at 15000 g (centrifuge AK-30, Sigma, Germany). The white peptide pallet was dissolved in 10 ml of distilled water and freeze dried.

The crude peptides were purified using HPLC on a Beckman Gold System (Beckman, USA) with an RP Kromasil-100, C8, 5 μm column (8×250 mm) (Knauer, Germany). The solvent systems were 0.1% TFA (A) and 80% acetonitrile in A (B). Either isocratic conditions or a linear gradient were applied (flow rate 3.0 mL/min, monitored at 226 nm). The purity of the synthesized peptides was checked on an RP Kromasil 100, C8, 5 μm column (4.6×250 mm) (Knauer, Germany). The solvent system was as above. Linear gradient from 10% to 90% B for 30 min, flow rate 1 mL/min, monitored at 226 nm. The mass spectrometry analysis was carried out on a MALDI MS (a Biflex III MALDI-TOF spectrometer, Bruker Daltonics, Germany) using a-CCA matrix.

HRMS: calcd for (MH⁺), 791.6528. found, 791.6584.

HNMR ¹H NMR (DMSO-d₆, ppm): δ 11.03 (s, 1H), 8.08 (s, 3H), 7.39-7.01 (m, 12H), 5.28 (s, 2H), 3.94-2.43 (m, 4H), 2.11-2.02 (m, 8H) 1.84-1.03 (m, 12H)

### Example 9

This example describes the synthesis of compound 9 Ac-Ahx-Pro-Tyr-Asp-Ala^{P}(O—C₆H₄-4-Cl)₂.

Ac-Ahx-Pro-Tyr-Asp-Ala^{P}(O—C₆H₄-4-Cl)₂ was synthesized manually following the solid-phase method using Fmoc chemistry. The following amino acid derivatives were used: Fmoc-Pro, Fmoc-Tyr(tBu), Fmoc-Asp(OtBu) along with N-acetyl-aminohaxanoic acid. The protected derivative of C-terminal amino acid residue, Fmoc-Asp(OtBu) 1.46 mmol, was attached to the 0.25 g 2-chlorotritylchloride resin (substitution of Cl 1.46 mequiv/g) (Calbiochem-Novabiochem AG, Switzerland) in the presence of an equimolar amount of DIPEA (based on the amino acid) under anhydrous conditions in 5 ml of DCM solution. The final effective substitution was determined spectrophotometrically and was equal to 0.82 mequiv/g). Peptide chains were elongated in consecutive cycles of deprotection and coupling. Deprotection was performed with 5 ml of 20% piperidine in DMF/NMP (1:1, v/v) with the addition of 1% Triton X-100, whereas chain elongation was achieved with standard DIC/HOBt chemistry; 0.36 mmol of each protected amino acid derivatives; Fmoc-Tyr(tBu) followed by Fmoc-Pro were used. The introduction of N-terminal N-actyl-aminohaexanoic acid was obtained by using 5 fold molar (1.8 mmol) excess of N-acetyl-aminohaxanoic acid and DIPCI in 2 ml anhydrous DMF and whole mixture was stirred for 2 hours at 30 C. The whole procedure was repeated 2 times. After completing the syntheses, the peptides were cleaved from the resin with 10 ml of TFE:DCM:acetic acid mixture (2:6:2, v/v/v).

0.3 mmoles of fully protected peptide was dissolved in 2 ml DMF and its carboxyl group was activated using equimolar amount of DIPCI. To such mixture the 0.5 mL of 0.31 mmoles of HBr×H₂N-Ala^{P}(O—C₆H₄-4-Cl)₂ in DMF and 0.31 mmol of DIPEA was added. The mixture was stirred for 6 hours and after that DMF was evaporated under reduced pressure. The obtained compound was suspended in 4 ml of TFA/phenol/triisopropylsilane/H₂O (88:5:2:5, v/v/v/v) mixture for 2 hours in order to removed side chain protection groups. The crude peptide was precipitate with anhydrous diethyl ether and centrifuged at 15000 g (centrifuge AK-30, Sigma, Germany). The white peptide pallet was dissolved in 10 ml of distilled water and freeze dried.

The crude peptides were purified using HPLC on a Beckman Gold System (Beckman, USA) with an RP Kromasil-100, C8, 5 μm column (8×250 mm) (Knauer, Germany). The solvent systems were 0.1% TFA (A) and 80% acetonitrile in A (B). Either isocratic conditions or a linear gradient were applied (flow rate 3.0 mL/min, monitored at 226 nm). The purity of the synthesized peptides was checked on an RP Kromasil 100, C8, 5 μm column (4.6×250 mm) (Knauer, Germany). The solvent system was as above. Linear gradient from 10% to 90% B for 30 min, flow rate 1 mL/min, monitored at 226 nm. The mass spectrometry analysis was carried out on a MALDI MS (a Biflex III MALDI-TOF spectrometer, Bruker Daltonics, Germany) using a-CCA matrix.

HRMS: calcd for (MH⁺), 876.7212. found, 876.8146.

HNMR ¹H NMR (DMSO-d₆, ppm): δ 11.03 (s 1H), 8.08 (s, 3H), 7.37-6.70 (m, 12H), 5.27 (s, 1H), 4.82-3.12 (m, 4H), 3.00-2.26 (m, 7H), 2.01-2.12 (m, 12H) 1.84-1.47 (m, 9H)

### Example 10

This example describes the synthesis of compound 10 ⁺H₂N-Pro-Tyr-Asp-Ala^{P}(O—C₆H₄-4-Cl)₂.

H-Pro-Tyr-Asp-Ala^{P}(O—C₆H₄-4-Cl)₂ was synthesized manually following the solid-phase method using Fmoc chemistry. The following amino acid derivatives were used: Boc-Pro, Fmoc-Tyr(tBu), Fmoc-Asp(OtBu). The protected derivative of C-terminal amino acid residue, Fmoc-Asp(OtBu) 1.46 mmol, was attached to the 0.25 g 2-chlorotritylchloride resin (substitution of Cl 1.46 mequiv/g) (Calbiochem-Novabiochem AG, Switzerland) in the presence of an equimolar amount of DIPEA (based on the amino acid) under anhydrous conditions in 5 ml of DCM solution. The final effective substitution was determined spectrophotometrically and was equal to 0.82 mequiv/g). Peptide chains were elongated in consecutive cycles of deprotection and coupling. Deprotection was performed with 5 ml of 20% piperidine in DMF/NMP (1:1, v/v) with the addition of 1% Triton X-100, whereas chain elongation was achieved with standard DIC/HOBt chemistry; 0.36 mmol of each protected amino acid derivatives; Fmoc-Tyr(tBu) followed by Boc-Pro were used. After completing the syntheses, the peptides were cleaved from the resin with 10 ml of TFE:DCM:acetic acid mixture (2:6:2, v/v/v).

0.3 mmoles of fully protected peptide was dissolved in 2 ml DMF and its carboxyl group was activated using equimolar amount of DIPCI. To such mixture the 0.5 mL of 0.31 mmoles of HBr×H₂N-Ala^{P}(O—C₆H₄-4-Cl)₂ in DMF and 0.31 mmol of DIPEA was added. The mixture was stirred for 6 hours and after that DMF was evaporated under reduced pressure. The obtained compound was suspended in 4 ml of TFA/phenol/triisopropylsilane/H₂O (88:5:2:5, v/v/v/v) mixture for 2 hours in order to removed side chain protection groups. The crude peptide was precipitated with anhydrous diethyl ether and centrifuged at 15000 g (centrifuge AK-30, Sigma, Germany). The white peptide pallet was dissolved in 10 ml of distilled water and freeze dried.

The crude peptides were purified using HPLC on a Beckman Gold System (Beckman, USA) with an RP Kromasil-100, C8, 5 μm column (8×250 mm) (Knauer, Germany). The solvent systems were 0.1% TFA (A) and 80% acetonitrile in A (B). Either isocratic conditions or a linear gradient were applied (flow rate 3.0 mL/min, monitored at 226 nm). The purity of the synthesized peptides was checked on an RP Kromasil 100, C8, 5 μm column (4.6×250 mm) (Knauer, Germany). The solvent system was as above. Linear gradient from 10% to 90% B for 30 min, flow rate 1 mL/min, monitored at 226 nm. The mass spectrometry analysis was carried out on a MALDI MS (a Biflex III MALDI-TOF spectrometer, Bruker Daltonics, Germany) using a-CCA matrix.

HRMS: calcd for (MH⁺), 721.5261. found, 722.5464.

HNMR ¹H NMR (DMSO-d₆, ppm): δ 11.03 (s 1H), 8.08 (s, 3H), 7.37-7.12 (m, 12H), 4.92-4.86 (m, 4H), 3.69-2.66 (m, 9H), 2.01-1.54 (m, 8H) 1.3 (d, 3H)

### Example 11

This example describes the synthesis of compound 11 Ac-Trp-Pro-Tyr-Asp-Ala^{P}(O—C₆H₄-4-Cl)₂.

Ac-Trp-Pro-Tyr-Asp-Ala^{P}(O—C₆H₄-4-Cl)₂ was synthesized manually following the solid-phase method using Fmoc chemistry. The following amino acid derivatives were used: Fmoc-Pro, Fmoc-Tyr(tBu), Fmoc-Asp(OtBu), Ac-Trp(Boc). The protected derivative of C-terminal amino acid residue, Fmoc-Asp(OtBu) 1.46 mmol, was attached to the 0.25 g 2-chlorotritylchloride resin (substitution of Cl 1.46 mequiv/g) (Calbiochem-Novabiochem AG, Switzerland) in the presence of an equimolar amount of DIPEA (based on the amino acid) under anhydrous conditions in 5 ml of DCM solution. The final effective substitution was determined spectrophotometrically and was equal to 0.82 mequiv/g). Peptide chains were elongated in consecutive cycles of deprotection and coupling. Deprotection was performed with 5 ml of 20% piperidine in DMF/NMP (1:1, v/v) with the addition of 1% Triton X-100, whereas chain elongation was achieved with standard DIC/HOBt chemistry; 0.36 mmol of each protected amino acid derivatives; Fmoc-Tyr(tBu) followed by, Fmoc-Pro and Ac-Trp(Boc) were used. After completing the syntheses, the peptides were cleaved from the resin with 10 ml of TFE:DCM:acetic acid mixture (2:6:2, v/v/v).

0.3 mmoles of fully protected peptide was dissolved in 2 ml DMF and its carboxyl group was activated using equimolar amount of DIPCI. To such mixture the 0.5 mL of 0.31 mmoles of HBr×H₂N-Ala^{P}(O—C₆H₄-4-Cl)₂ in DMF and 0.31 mmol of DIPEA was added. The mixture was stirred for 6 hours and after that DMF was evaporated under reduced pressure. The obtained compound was suspended in 4 ml of TFA/phenol/triisopropylsilane/H₂O (88:5:2:5, v/v/v/v) mixture for 2 hours in order to removed side chain protection groups. The crude peptide was precipitate with anhydrous diethyl ether and centrifuged at 15000 g (centrifuge AK-30, Sigma, Germany). The white peptide pallet was dissolved in 10 ml of distilled water and freeze dried.

The crude peptides were purified using HPLC on a Beckman Gold System (Beckman, USA) with an RP Kromasil-100, C8, 5 μm column (8×250 mm) (Knauer, Germany). The solvent systems were 0.1% TFA (A) and 80% acetonitrile in A (B). Either isocratic conditions or a linear gradient were applied (flow rate 3.0 mL/min, monitored at 226 nm). The purity of the synthesized peptides was checked on an RP Kromasil 100, C8, 5 μm column (4.6×250 mm) (Knauer, Germany). The solvent system was as above. Linear gradient from 10% to 90% B for 30 min, flow rate 1 mL/min, monitored at 226 nm. The mass spectrometry analysis was carried out on a MALDI MS (a Biflex III MALDI-TOF spectrometer, Bruker Daltonics, Germany) using a-CCA matrix.

HRMS: calcd for (MH⁺), 949.7728. found, 949.8251.

HNMR ¹H NMR (DMSO-d₆, ppm): δ 11.03 (s 1H), 10.10 (s, 1H), 8.08 (s, 3H), 7.47-6.83 (m, 17H), 5.27 (s, 1H), 4.92-4.37 (m, 5H), 3.44-2.54 (m, 10H), 2.11-2.01 (m, 2H) 1.84-1.47 (m, 6H)

### Example 12

This example describes the synthesis of compound 12 Ac-Ile-Pro-Tyr-Asp-Ala^{P}(O—C₆H₄-4-Cl)₂.

Ac-Ile-Pro-Tyr-Asp-Ala^{P}(O—C₆H₄-4-Cl)₂ was synthesized manually following the solid-phase method using Fmoc chemistry. The following amino acid derivatives were used: Ac-Ile, Fmoc-Pro, Fmoc-Tyr(tBu), Fmoc-Asp(OtBu). The protected derivative of C-terminal amino acid residue, Fmoc-Asp(OtBu) 1.46 mmol, was attached to the 0.25 g 2-chlorotritylchloride resin (substitution of Cl 1.46 mequiv/g) (Calbiochem-Novabiochem AG, Switzerland) in the presence of an equimolar amount of DIPEA (based on the amino acid) under anhydrous conditions in 5 ml of DCM solution. The final effective substitution was determined spectrophotometrically and was equal to 0.82 mequiv/g). Peptide chains were elongated in consecutive cycles of deprotection and coupling. Deprotection was performed with 5 ml of 20% piperidine in DMF/NMP (1:1, v/v) with the addition of 1% Triton X-100, whereas chain elongation was achieved with standard DIC/HOBt chemistry; 0.36 mmol of each protected amino acid derivatives; Fmoc-Tyr(tBu) followed by, Fmoc-Pro and Ac-Ile were used. After completing the syntheses, the peptides were cleaved from the resin with 10 ml of TFE:DCM:acetic acid mixture (2:6:2, v/v/v).

0.3 mmoles of fully protected peptide was dissolved in 2 ml DMF and its carboxyl group was activated using equimolar amount of DIPCI. To such mixture the 0.5 mL of 0.31 mmoles of HBr×H₂N-Ala^{P}(O—C₆H₄-4-Cl)₂ in DMF and 0.31 mmol of DIPEA was added. The mixture was stirred for 6 hours and after that DMF was evaporated under reduced pressure. The obtained compound was suspended in 4 ml of TFA/phenol/triisopropylsilane/H₂O (88:5:2:5, v/v/v/v) mixture for 2 hours in order to removed side chain protection groups. The crude peptide was precipitate with anhydrous diethyl ether and centrifuge at 15000 g (centrifuge AK-30, Sigma, Germany). The white peptide pallet was dissolved in 10 ml of distilled water and freeze dried.

The crude peptides were purified using HPLC on a Beckman Gold System (Beckman, USA) with an RP Kromasil-100, C8, 5 μm column (8×250 mm) (Knauer, Germany). The solvent systems were 0.1% TFA (A) and 80% acetonitrile in A (B). Either isocratic conditions or a linear gradient were applied (flow rate 3.0 mL/min, monitored at 226 nm). The purity of the synthesized peptides was checked on an RP Kromasil 100, C8, 5 μm column (4.6×250 mm) (Knauer, Germany). The solvent system was as above. Linear gradient from 10% to 90% B for 30 min, flow rate 1 mL/min, monitored at 226 nm. The mass spectrometry analysis was carried out on a MALDI MS (a Biflex III MALDI-TOF spectrometer, Bruker Daltonics, Germany) using a-CCA matrix.

HRMS: calcd for (MH⁺), 876.7259. found, 876.7342.

HNMR ¹H NMR (DMSO-d₆, ppm): δ 11.03 (s 1H), 8.08 (s, 3H), 7.33-6.68 (m, 12H), 5.27 (s, 1H), 4.82-4.40 (m, 5H), 3.44-2.46 (m, 6H), 2.12-1.98 (m, 3H) 1.84-1.03 (m, 11H)

### Example 13

This example describes the synthesis of compound 13 Biotin-PEG₃₀₀₀-Pro-Tyr-Asp-Ala^{P}(O—C₆H₄-4-Cl)₂.

Biotin-PEG₃₀₀₀-Pro-Tyr-Asp-Ala^{P}(Oph-p-Cl)₂ was synthesized manually following the solid-phase method using Fmoc chemistry. The following amino acid derivatives were used: Fmoc-Pro, Fmoc-Tyr(tBu), Fmoc-Asp(OtBu). The protected derivative of C-terminal amino acid residue, Fmoc-Asp(OtBu) 1.46 mmol, was attached to the 0.25 g 2-chlorotritylchloride resin (substitution of Cl 1.46 mequiv/g) (Calbiochem-Novabiochem AG, Switzerland) in the presence of an equimolar amount of DIPEA (based on the amino acid) under anhydrous conditions in 5 ml of DCM solution. The final effective substitution was determined spectrophotometrically and was equal to 0.82 mequiv/g). Peptide chains were elongated in consecutive cycles of deprotection and coupling. Deprotection was performed with 5 ml of 20% piperidine in DMF/NMP (1:1, v/v) with the addition of 1% Triton X-100, whereas chain elongation was achieved with standard DIC/HOBt chemistry; 0.36 mmol of each protected amino acid derivatives; Fmoc-Tyr(tBu) followed by Fmoc-Pro were used. Next the alpha-(9-Fluorenylmethyloxycarbonyl)amino-omega-carboxypoly(ethylene glycol) Fmoc-PEG₃₀₀₀ (MW 3000) was coupled to the amino group of Pro. The introduction of N-terminal biotin group was obtained by using 5 fold molar (1.8 mmol) excess of biotin and DIPCI in 2 ml anhydrous DMSO and whole mixture was stirred for 6 hour at 30 C. The whole procedure was repeated 2 times. After completing the syntheses, the peptides were cleaved from the resin with 10 ml of TFE:DCM:acetic acid mixture (2:6:2, v/v/v).

0.3 mmoles of fully protected peptide was dissolved in 2 ml DMF and its carboxyl group was activated using equimolar amount of DIPCI. To such mixture the 0.5 mL of 0.31 mmoles of HBr×H₂N-Ala^{P}(O—C₆H₄-4-Cl)₂ in DMF and 0.31 mmol of DIPEA was added. The mixture was stirred for 6 hours and after that DMF was evaporated under reduced pressure. The obtained compound was suspended in 4 ml of TFA/phenol/triisopropylsilane/H₂O (88:5:2:5, v/v/v/v) mixture for 2 hours in order to removed side chain protection groups. The crude peptide was precipitate with anhydrous diethyl ether and centrifuged at 15000 g (centrifuge AK-30, Sigma, Germany). The white peptide pallet was dissolved in 10 ml of distilled water and freeze dried.

The crude peptides were purified using HPLC on a Beckman Gold System (Beckman, USA) with an RP Kromasil-100, C8, 5 μm column (8×250 mm) (Knauer, Germany). The solvent systems were 0.1% TFA (A) and 80% acetonitrile in A (B). Either isocratic conditions or a linear gradient were applied (flow rate 3.0 mL/min, monitored at 226 nm). The purity of the synthesized peptides was checked on an RP Kromasil 100, C8, 5 μm column (4.6×250 mm) (Knauer, Germany). The solvent system was as above. Linear gradient from 10% to 90% B for 30 min, flow rate 1 mL/min, monitored at 226 nm. The mass spectrometry analysis was carried out on a MALDI MS (a Biflex III MALDI-TOF spectrometer, Bruker Daltonics, Germany) using a-CCA matrix.

HRMS: calcd for (MH⁺), 4824.0648. found (4823.1021).

HNMR ¹H NMR (DMSO-d₆, ppm): δ 11.03 (s 1H), 8.08 (s, 3H), 7.37-7.12 (m, 12H), 5.98 (s, 2H) 5.27 (s, 1H), 4.92-4.37 (m, 4H), 3.74-2.66 (m, 400H), 2.01-2.12 (m, 8H) 1.84-1.47 (m, 6H)

### Results

### Design of the Peptidyl Recognition Sequence

Solvent accessible surfaces in HNE and PR3 differ significantly by their charge distribution in the vicinity of their substrate binding site (reviewed in (4)) S2 pocket in PR3 is smaller and more hydrophilic than that in HNE mainly because of the substitution of a Leu by a Lys at position 99 in the vicinity of both the S2 and S4 pockets (4). Another critical difference between the two proteases is the replacement of a charged arginyl residue at position 217 in HNE by an Ile in PR3 in the vicinity of the S4 subsite. We investigated the importance of the S4 subsite in PR3 by producing two recombinant PR3 molecules with a Lys for Leu substitution at position 99 (PR3K₉₉L) and an Ile for Arg substitution at position 217 (PR3I₂₁₇R). Wild type and recombinant PR3 were then assayed with FRET substrates derived from the PR3/NE substrate Abz-QPMAVVQSVPQ-EDDnp, and bearing either a negatively charged residue (Abz-QDMAVVQSVPQ-EDDnp) or a positively charged residue (QKMAVVQSVPQ-EDDnp) at P4. Wild type and recombinant proteases all cleave the original substrate at the V—V bond with about the same kcat/Km (Table 2).

TABLE 2
Enzymatic activity of PR3 and their mutants

| | wtPR3 | PR3I₂₁₇R | PR3K₉₉L | HNE |
|---|---|---|---|---|
| FRET SUBSTRATES | k_{cat}/K_{M} (mM⁻¹s⁻¹) | | | |
| ABZ-Gln-Pro-Met-Ala-Val-Val-Gln-Ser-Val-Pro- | 95.7 | 86.6 | 85.7 | 149 |
| Gln-EDDnp | | | | |
| ABZ-Gln-Asp-Met-Ala-Val-Val-Gln-Ser-Val-Pro- | 1.6 | 194.3 | 0.46 | 77 |
| Gln-EDDnp | | | | |
| ABZ-Gln-Lys-Met-Ala-Val-Val-Gln-Ser-Val- | 74.6 | 72 | 60.6 | 210 |
| Pro-Gln-EDDnp | | | | |
| ABZ-Gln-Pro-Met-Asp-Val-Val-Gln-Ser-Val-Pro- | 1001 | 221 | <2 | 119 |
| Gln-EDDnp | | | | |
| ABZ-Gln-Asp-Met-Asp-Val-Val-Gln-Ser-Val-Pro- | 1.7 | 557 | 2.1 | 86 |
| Gln-EDDnp | | | | |

| | |
|---|---|
| wtPR3 | HNE |

| | | |
|---|---|---|
| PARANILIDE SUBSTRATES | [S] mM | k_{cat}/K_{M} (M⁻¹s⁻¹) |

| | | | |
|---|---|---|---|
| Ac-Pro-Tyr-Asp-Ala-pNA | 1 | 4201 ± 29.7 | n.h |
| Ac-Asp-Tyr-Asp-Ala-pNA | 2 | 162 ± 12.8 | n.h |

But the kcat/Km for the hydrolysis of Abz-QDMAVVQSVPQ-EDDnp decreases by a factor 3 using wtPR3 and is 6 times larger using PR3I₂₁₇R, whereas it completely resists cleavage by PR3K99L. The cleavage site at the V—V bond remained unchanged using PR3I₂₁₇R. Interestingly, HNE cleaved Abz-QDMAVVQSVPQ-EDDnp at the same V-Q bond as that we previously identified in Abz-QPMDVVQSVPQ-EDDnp which further illustrates that that NE, unlike PR3, cannot accommodate a negative charge at P2 and P4. Introducing a positive charge at P4 does not significantly change the kcat/Km towards wild type and recombinant PR3. We deduced from these data that the sequence DxDA would be the most appropriate to construct a PR3-selective inhibitor of high affinity. We observed that a large panel of amino acid residues could be accommodated at the PR3 P3 subsite using a series of FRET substrates but a Tyr was preferred at this position (Table 3).

TABLE 3
P3 specificity for proteinase 3

| | k_{cat}/Kₘ |
|---|---|
| | Proteinase 3 |
| Substrates | mM⁻¹ · s⁻¹ |
| Abz-FTFRSARQ-EDDnp | 36 |
| Abz-FTFLSARQ-EDDnp | 17 |
| Abz-FTFYSARQ-EDDnp | 110 |

Then we used the peptidyl sequences DYDA- and PYDA- to construct two paranitroanlide substrates bearing this sequence from P4 to P1. Ac-DYDA-pNA and Ac-PYDA-pNA were assayed with purified PR3 and HNE and the specificity constants kcat/Km were determined. We found that HNE did not cleave these substrates whereas they were rapidly cleaved by PR3. We then constructed phosphonate inhibitors with the same peptidyl sequence i.e. Ac-DYDA^{P}(Oph-p-Cl)₂ and Ac-PYDA^{P}(Oph-p-Cl)₂.

Synthesis and Inhibitory Properties of Ac-Peptidyl-P(OPh-p-Cl)₂ Phosphonate Inhibitors and their Biotinylated and Fluorescent Derivatives

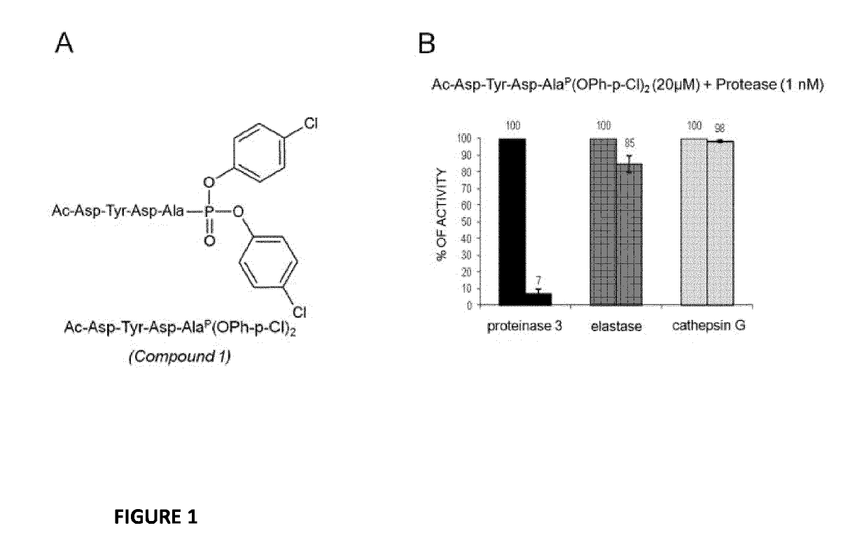

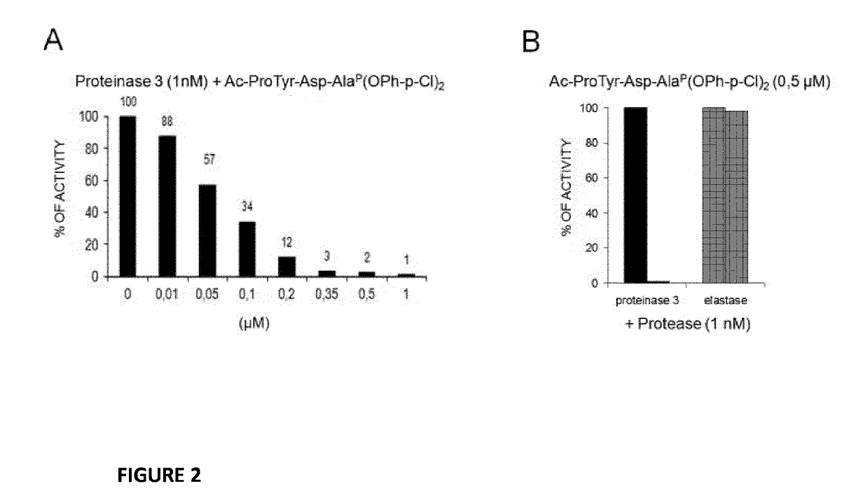

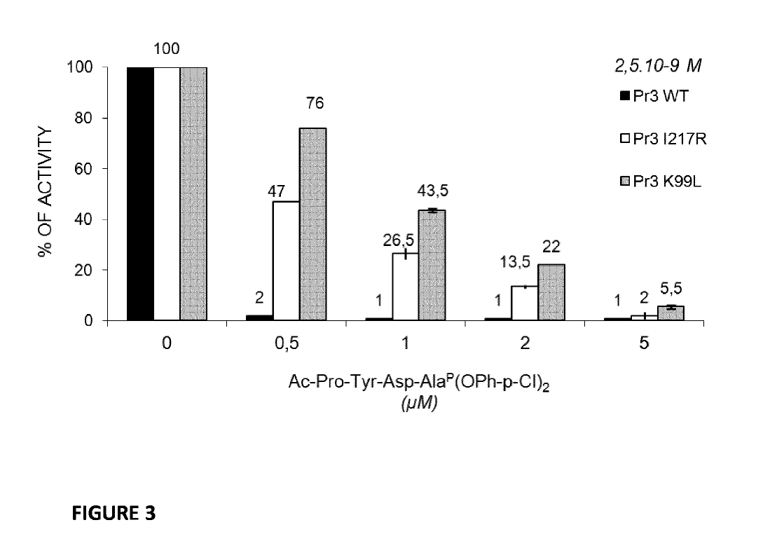

Phosphonate inhibitors and their biotinylated and fluorescent derivatives were synthesized, purified and controlled by mass spectrometry as reported. Their purity was checked by HPLC (data not shown). Purified Ac-Asp-Tyr-Asp-Ala^{P}(O—C₆H₄-4-Cl)₂ (compound 1) and Ac-Pro-Tyr-Asp-Ala^{P}(O—C₆H₄-4-Cl)₂ (compound 2) were first assayed for their inhibitory properties by measuring the residual activity of the human neutrophil serine proteases PR3, HNE and CatG in the presence of increasing amounts of each compound. We used for this purpose the FRET substrates that we developed previously for each protease (18). An almost complete inhibition of PR3 (1 nM final) was obtained after a 30 min incubation time with 20 μM of compound 1 (FIG. 1) and 0.3 μM of compound 2 (FIG. 2). Recombinant PR3K₉₉L, PR3I₂₁₇L, HNE and CatG were used in the same experimental conditions. We observed no significant inhibition of HNE (1 nM) (FIG. 2) and CatG (1 nM) (not shown). The replacement in PR3K₉₉L of Lys₉₉ in the S2 subsite of PR3 by the leucyl residue present in HNE made this mutant far less sensitive to inhibition by both compounds (FIG. 3). This was also observed by replacing Ile₂₁₇ in the S4 subsite of wt PR3 by its HNE homologue Arg₂₁₇ though both inhibitors were more efficient toward this mutant (FIG. 3). This emphasizes the importance of the S2 and S4 subsites to discriminate between PR3 and HNE specificities. Significant inhibition of HNE was obtained using about 100 times more Ac-Pro-Tyr-Asp-Ala^{P}(O—C₆H₄-4-Cl)₂ than required to inhibit PR3. This concentration in inhibitor however compares with that of compound 1 used to selectively inhibit the same amount of PR3 which demonstrates that compound 1 is less potent but more selective than compound 2. We confirmed this observation kinetically by measuring the second order rate constants kobs/l by competition experiments between the PR3 substrate and each inhibitor (17). As expected the second order rate constant kobs/l for compound 2 was about 100 times greater than that for compound 1 (see Table 1).

We then replaced the N-terminal acetyl group by biotin in compounds 1 and 2 to use the resulting compounds (named 3 and 4 respectively), as molecular probes to identify PR3 in soluble biological samples, or in cells and tissues or by a fluorescent group. The presence of the biotin moiety at P₅ significantly improved the kobs/I for both inhibitors (Table 1). The kobs/I for Biotin-Pro-Tyr-Asp-Ala^{P}(O—C₆H₄-4-Cl)₂ was in the 10³M⁻¹s⁻¹ range. The Biotin/acetyl change at P₅ significantly and cumulatively improved the interaction between inhibitors and PR3, the results thus demonstrate the importance of an extended active site that allows to discriminate between the activities of PR3 and HNE. We then replaced the biotin group by other hydrophobic groups, such as linear CH₃—(CH₂)₄ alkyl, N-acetyl aminohexanoic chain (respectively compound 8 and 9, see Table 1). We confirm that the presence of such hydrophic groups, similarly to biotin, significantly increase the kobs/I to the 10³M-1s-1 range. As discussed below, hydrophobic groups most probably helps to stabilize the inhibitor within the PR3 active site. In contrast, compounds with ammonium or Trp or Ile amino acids at P5 position did not significantly increase the kobs/I (respectively compounds 10, 11 and 12, see Table 1).

Biotin may be linked to the peptidyl moiety of the inhibitor via a spacer selected from the group consisting of PEG (e.g. PEG3000, see Compound 13, Table 1) to improve its recognition by extravidin-peroxydase and visualize PR3 at the cell surface or inside permeabilized cells.

We then investigated whether these phosphonate inhibitors were efficient for inhibiting PR3 in a complex biological environment and to resist degradation by other proteases

### Inhibition of PR3 by Phosphonate Inhibitors in a Human Neutrophil Lysate and in Inflammatory Biological Samples

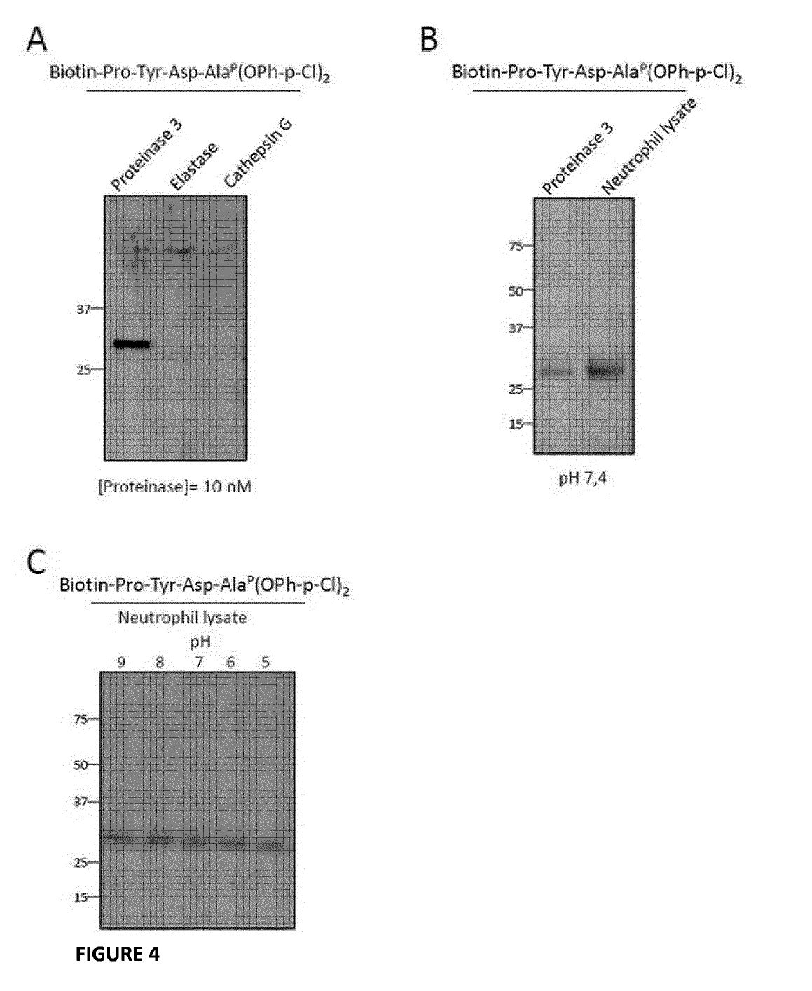

We first studied the stability and the selectivity of compound 1, 2 and their biotinylated derivatives in a lysate of purified human neutrophils. The PR3 activity was totally inactivated by all inhibitors in experimental conditions reproducing those used above with purified proteases (FIG. 4). Further, PR3 was selectively inactivated in the neutrophil lysate as shown by western blotting using biotinylated inhibitors. No loss of the inhibition potential was observed after a one hour preincubation of the neutrophil lysate-inhibitor mixture before adding the substrates, which shows that inhibitors resisted degradation in an environment replete with proteases (not shown).

We then studied the fate of inhibitors in a neutrophil suspension to know whether they interact with the cell membrane and/or they enter the cell as it was shown before with phosphonate inhibitors of CatG (19). The concentration of compound with fluorescent moiety but not that of other inhibitors decreased significantly in the supernatant of a suspension of freshly purified, quiescent neutrophils that show minimal PR3 activity. Since we observed that phosphonate inhibitors resisted degradation in a neutrophil lysate, this means that it has interacted with the cell surface or has entered the cells (data not shown). We checked the possible cell permeability of compound with fluorescent moiety, by measuring PR3 activity in the supernatant fraction of purified, quiescent neutrophils first incubated for 1 hour with the inhibitor, then washed and lysed in buffer and we compared this activity with that measured using naïve cells.

Crude lung secretions of patients with chronic inflammatory disorders associating significant neutrophil recruitment (IPF, Cystic fibrosis) were used as biological material to assay the efficacy of phosphonate inhibitors. We first measured the rate of cleavage of the PR3 substrate in >3 IPF and >3 CF crude samples before and after incubation with compounds 2 and 4 (×μM final). All crude samples cleaved the PR3 substrate in the absence of inhibitor and the cleavage always occurred at the expected bond, i.e. the cleavage bond of PR3, as checked by HPLC. The PR3 concentration was in the 0.1-1 μM range in lung secretions, based on the rate of substrate hydrolysis. We observed a total inhibition of the substrate hydrolysis after a 30 min incubation time with either compound 2 or its biotinylated derivative (compound 4) whereas HNE and CatG activities remained unchanged. Further, the rate of PR3 inactivation in a crude biological sample compared with that of purified PR3, which means that there is no adverse effect impairing the PR3 inhibition in lung secretions of patients with inflammatory disorders.

### REFERENCES

1. Turk, B. (2006) Targeting proteases: successes, failures and future prospects. Nat Rev Drug Discov 5, 785-799
2. Kallenberg, C. G. (2008) Pathogenesis of PR3-ANCA associated vasculitis. J Autoimmun 30, 29-36
3. Kallenberg, C. G., Heeringa, P., and Stegeman, C. A. (2006) Mechanisms of Disease: pathogenesis and treatment of ANCA-associated vasculitides. Nat Clin Pract Rheumatol 2, 661-670
4. Korkmaz, B., Horwitz, M., Jenne, D. E., and Gauthier, A. (2010) Neutrophil elastase, proteinase 3 and cathepsin G as therapeutic targets in human diseases. Pharmacol Rev 62
5. Pendergraft, W. F., 3rd, Rudolph, E. H., Falk, R. J., Jahn, J. E., Grimmler, M., Hengst, L., Jennette, J. C., and Preston, G. A. (2004) Proteinase 3 sidesteps caspases and cleaves p21(Waf1/Cip1/Sdi1) to induce endothelial cell apoptosis. Kidney Int 65, 75-84
6. Preston, G. A., Zarella, C. S., Pendergraft, W. F., 3rd, Rudolph, E. H., Yang, J. J., Sekura, S. B., Jennette, J. C., and Falk, R. J. (2002) Novel effects of neutrophil-derived proteinase 3 and elastase on the vascular endothelium involve in vivo cleavage of NF-kappaB and proapoptotic changes in JNK, ERK, and p38 MAPK signaling pathways. J Am Soc Nephrol 13, 2840-2849
7. Korkmaz, B., Lesner, A., Letast, S., Mandi, Y. K., Jourdan, M. L., Dallet-Choisy, S., Marchand-Adam, S., Kellenberger, C., Viaud-Massuard, M. C., Jenne, D. E., and Gauthier, F. (2013) Neutrophil proteinase 3 and dipeptidyl peptidase I (cathepsin C) as pharmacological targets in granulomatosis with polyangiitis (Wegener gran ulomatosis). Semin Immunopathol 35, 411-421
8. Schreiber, A., Busjahn, A., Luft, F. C., and Kettritz, R. (2003) Membrane expression of proteinase 3 is genetically determined. J Am Soc Nephrol 14, 68-75.
9. Korkmaz, B., Kellenberger, C., Viaud-Massuard, M. C., and Gauthier, F. (2013) Selective inhibitors of human neutrophil proteinase 3. Curr Pharm Des
10. Grzywa, R., and Sienczyk, M. (2013) Phosphonic esters and their application of protease control. Curr Pharm Des 19, 1154-1178
11. Boduszek, B., Brown, A. D., and Powers, J. C. (1994) alpha-Aminoalkylphosphonate di(chlorophenyl) esters as inhibitors of serine proteases. J Enzyme Inhib 8, 147-158
12. Brown, C. M., Ray, M., Eroy-Reveles, A. A., Egea, P., Tajon, C., and Craik, C. S. Peptide length and leaving-group sterics influence potency of peptide phosphonate protease inhibitors. Chem Biol 18, 48-57
13. Reich, M., Lesner, A., Legowska, A., Sienczyk, M., Oleksyszyn, J., Boehm, B. O., and Burster, T. (2009) Application of specific cell permeable cathepsin G inhibitors resulted in reduced antigen processing in primary dendritic cells. Mol Immunol 46, 2994-2999
14. Kam, C. M., Kerrigan, J. E., Dolman, K. M., Goldschmeding, R., Von dem Borne, A. E., and Powers, J. C. (1992) Substrate and inhibitor studies on proteinase 3. FEBS Lett 297, 119-123.
15. Sienczyk, M., and Oleksyszyn, J. (2006) Inhibition of trypsin and urokinase by Cbz-amino(4-guanidinophenyl)methanephosphonate aromatic ester derivatives: the influence of the ester group on their biological activity. Bioorg Med Chem Lett 16, 2886-2890
16. Oleksyszyn, J., Subotkowska, L., and Mastalerz, P. (1979) Synthesis 985
17. Tian, W. X., and Tsou, C. L. (1982) Determination of the rate constant of enzyme modification by measuring the substrate reaction in the presence of the modifier. Biochemistry 21, 1028-1032
18. Korkmaz, B., Attucci, S., Juliano, M. A., Kalupov, T., Jourdan, M. L., Juliano, L., and Gauthier, F. (2008) Measuring elastase, proteinase 3 and cathepsin G activities at the surface of human neutrophils with fluorescence resonance energy transfer substrates. Nat Protoc 3, 991-1000
19. Woodard, S. L., Jackson, D. S., Abuelyaman, A. S., Powers, J. C., Winkler, U., and Hudig, D. (1994) Chymase-directed serine protease inhibitor that reacts with a single 30-kDa granzyme and blocks NK-mediated cytotoxicity. J Immunol 153, 5016-5025
20. Jegot, G. et al. (2011) A substrate-based approach to convert SerpinB1 into a specific inhibitor of proteinase 3, the Wegener's granulomatosis autoantigen. FASEB Journal 25, 3019-3031
21. Epinette C. et al. (2012) A selective reversible azapeptide inhibitor of human neutrophil proteinase 3 derived from a high affinity FRET substrate. Biochemical Pharmacology 83, 788-796

1. A peptidyl phosphonate ester compound having the structure of formula (I): 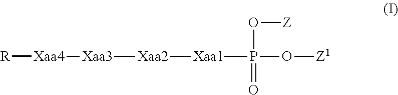
or a pharmaceutically acceptable salt thereof, wherein
Z and Z¹ are the same or different and are selected from the group consisting of C₁₋₆ perfluoralkyl, phenyl, phenyl substituted with J, phenyl disubstituted with J, phenyl trisubstituted with J, and, pentafluorophenyl, wherein J is selected from the group consisting of halogen, S, C₁₋₆ alkyl, C₁₋₆perfluoroalkyl, C₁₋₆ alkoxy, NO₂, CN, OH, CO₂H, amino, C₁₋₆alkylamino, C₂₋₁₂dialkylamino, C₁₋₆ acyl, and C₁₋₆alkoxy-CO—, and C₁₋₆alkly-S—,
R is absent, or is selected from the group consisting of a protecting group, a hydrophobic group and acetyl,
Xaa4 is an amino acid selected from the group consisting of proline, a hydrophobic amino acid and a negatively charged amino acid,
Xaa3 is not alanine and is selected from the group consisting of glycine, tyrosine, valine, leucine, isoleucine, proline, methionine, phenylalanine, tryptophane, serine, threonine, cysteine, asparagine, glutamine, aspartic acid, glutamic acid, lysine, arginine, histidine, phenylglycine, norleucine, norvaline, ornithine and citrulline,
Xaa2 is a negatively charged amino acid or is selected from the group consisting of tyrosine, serine, and phenylalanine, and
Xaa1 is alanine, valine or norvaline, or an equivalent non-polar and non-charged amino acid.

2. The peptidyl phosphonate ester compound of claim 1, wherein Z and Z¹ are selected from the group consisting of phenyl, phenyl substituted with J, and phenyl disubstituted with J.

3. The peptidyl phosphonate ester compound of claim 1, wherein R is selected from the group consisting of: (i) a non-polar aliphatic group or aryl group and their esters, ethers or polyethers; (ii) one or more hydrophobic amino acids selected from the group consisting of alanine, isoleucine, leucine, methionine, phenylalanine, proline, tryptophane and valine; and, (iii) glycine.

4. The peptidyl phosphonate ester compound of claim 1, wherein Xaa4 is proline.

5. The peptidyl phosphonate ester compound of claim 1, wherein Xaa3 is tyrosine.

6. The peptidyl phosphonate ester compound of claim 1, wherein Xaa2 is aspartic acid.

7. The peptidyl phosphonate ester compound of claim 1, wherein Xaa1 is alanine, valine or norvaline.

8. The peptidyl phosphonate ester compound of claim 1, wherein Z and Z1 are 4-chlorophenyl.

9. The peptidyl phosphonate ester compound of claim 1, which is selected from the group consisting of:
i. Ac-Asp-Tyr-Asp-Ala^{P}(O—C₆H₄-4-Cl)₂;
ii. Ac-Pro-Tyr-Asp-Ala^{P}(O—C₆H₄-4-Cl)₂;
iii. Biotin-Asp-Tyr-Asp-Ala^{P}(O—C₆H₄-4-Cl)₂;
iv. Biotin-Pro-Tyr-Asp-Ala^{P}(O—C₆H₄-4-Cl)₂;
v. Biotin-Pro-Tyr-Asp-Avl^{P}(O—C₆H₄-4-Cl)₂;
vi. Biotin-Val-Tyr-Asp-Ala^{P}(O—C₆H₄-4-Cl)₂
vii. Biotin-Val-Tyr-Asp-Avl^{P}(O—C₆H₄-4-Cl)₂
viii. Biotin-(PEG)-Pro-Tyr-Asp-Ala^{P}(O—C₆H₄-4-Cl)₂;
ix. Ac-Pro-Tyr-Phe-Ala^{P}(O—C₆H₄-4-Cl)₂;
x. CH₃(CH₂)₄—Pro-Tyr-Asp-Ala^{P}(O—C₆H₄-4-Cl)₂;
xi. Ac-Ahx-Pro-Tyr-Asp-Ala^{P}(O—C₆H₄-4-Cl)₂;
xii. ⁺H₂N-Pro-Tyr-Asp-Ala^{P}(O—C₆H₄-4-Cl)₂;
xiii. Ac-Trp-Pro-Tyr-Asp-Ala^{P}(O—C₆H₄-4-Cl)₂;
xiv. Ac-Ile-Pro-Tyr-Asp-Ala^{P}(O—C₆H₄-4-Cl)₂,
or their pharmaceutically acceptable salts.

10. The peptidyl phosphonate ester compound of claim 1, wherein said peptidyl phosphonate ester compound is a selective inhibitor of proteinase 3.

11. (canceled)

12. A method of treating disorders characterized by abnormal or pathological activity of proteinase 3 in a subject in need thereof, comprising administering to the subject a therapeutically effective amount of a compound of claim 1.

13. (canceled)

14. A pharmaceutical composition, comprising a peptidyl phosphonate ester compound according to claim 1, and one or more pharmaceutically acceptable excipients.

15. An in vitro method for detecting or quantifying proteinase 3 in a biological sample, comprising the steps of
i. providing a biological sample;
ii. contacting said biological sample with a peptidyl phosphonate ester compound of claim 1 under conditions for specific binding of said peptidyl phosphonate ester compound with proteinase 3; and,
iii. detecting specific binding of said peptidyl phosphonate ester compound with proteinase 3;
wherein said specific binding enables determining the presence or amount of proteinase 3 in said biological sample.

16. The peptidyl phosphonate ester compound of claim 1, wherein the hydrophobic amino acid is valine.

17. The peptidyl phosphonate ester compound of claim 3, wherein the non-polar aliphatic group is a linear or branched alkyl chain.

18. The peptidyl phosphonate ester compound of claim 17, wherein the linear or branched alkyl chain is a C5-C10 linear or branched alkyl chain.
